(19) United States
(12) Patent Application Publication
(10) Pub. No.: US 2026/0225889 A1
(43) Pub. Date: Aug. 6, 2026

(54) Spherical nanoparticles for improved composite materials

(71) Applicant: Stichting Radboud universitair medisch centrum, Nijmegen (NL)

(72) Inventors: Sander Cornelis Gerardus Leeuwenburgh, Nijmegen (NL); Rong Wang, Nijmegen (NL); Lizzy Anne Babs Cuypers, Nijmegen (NL)

(73) Assignee: Stichting Radboud universitair medisch centrum, Nijmegen (NL)

(21) Appl. No.: 19/159,622

(22) PCT Filed: Mar. 7, 2024
(86) PCT No.: PCT/EP2024/055978

§ 371 Date: Aug. 26, 2025

(30) Foreign Application Priority Data
Mar. 7, 2023 (EP) 23160581.7

(51) Int. Cl. C01B 25/32; A61K 9/06; A61K 33/30; A61K 38/14; A61K 47/02; A61K 47/69

(52) U.S. Cl. CPC C01B 25/327; A61K 9/06; A61K 33/30; A61K 38/14; A61K 47/02; A61K 47/6931; C01P 2004/32; C01P 2004/60; C01P 2006/90

(57) ABSTRACT

The invention relates to a method for preparing gels with improved mechanical properties, and to components useful in the manufacture of such a gel, and to the manufacture of such components. The gels are robust to mechanical attack and destructive shear, and comprise spherical nanoparticles.

### FIELD OF THE INVENTION

The invention relates to a method for preparing gels with improved mechanical properties, and to components useful in the manufacture of such a gel, and to the manufacture of such components. The gels are robust to mechanical attack and destructive shear, and comprise spherical nanoparticles.

### BACKGROUND ART

Colloidal gels are a particularly attractive class of hydrogels for applications in regenerative medicine, and allow for a “bottom-up” fabrication of multi-functional biomaterials by employing micro- or nanoscale particles as building blocks to assemble into shape-specific bulk scaffolds. So far, however, the synthesis of colloidal composite gels composed of both organic and inorganic particles has not been widely investigated.

Wang et al. (Acta Biomaterialia, 2013, doi: 10.1016/j.actbio.2013.08.036) described the development of injectable colloidal organic-inorganic composite gels using calcium phosphate (CaP) nanoparticles and gelatine (Gel) nanospheres as building blocks. These colloidal composite gels exhibited increased gel elasticity after addition of CaP nanoparticles, but shear-thinning and self-healing behaviour were not markedly improved. Gel stiffness was increased to a limited extent. The improvements in properties of these gels were achieved using needle-like calcium phosphate nanoparticles of high aspect ratio.

However, improvements are still desired regarding increased mechanical properties for hydrogels. There is a need for improved gel materials, featuring for instance improved elasticity, improved cohesion, and/or improved robustness to multiple shearings. There is a need for improved materials for use in gel formation. There is a need for improved methods for production of gels or for production of components useful for the formation of gels. There is a need for gels with improved robustness to mechanical attack or destructive shear.

Bone infections pose a significant challenge for healthcare, since they often lead to inflammatory destruction or ultimately necrosis of bone tissue. Such infections predominantly arise following trauma or surgical intervention, including the application of joint prostheses or orthopedic fixation devices. One of the most frequent causative pathogens of bone infections is Staphylococcus aureus. S. aureus is an opportunistic Gram-positive bacterial species that commonly resides in the human skin and nasal cavity. However, it has the potential to cause severe and life-threatening infections upon invasion of the bloodstream or bone tissue. Although S. aureus is traditionally recognized as an extracellular pathogen, increasing evidence highlights its ability to persist intracellularly inside mammalian cells such as macrophages. This intracellular survival creates a formidable challenge for current treatment strategies, since commonly used antibiotics are unable to cross the cell membrane (Barcia-Macay, M., et al., Antimicrobial agents and chemotherapy, 2006. 50(3): p. 841-851). Furthermore, once internalized, S. aureus enters a “persister” state characterized by reduced metabolic activity, thereby rendering S. aureus highly resistant to antibiotics, even at high concentrations. Consequently, intracellular S. aureus is shielded from antibiotics, which may induce recurrent and chronic bone infections.

Hence, there is a need to develop an antibacterial treatment that can effectively target and eliminate intracellular S. aureus. It is desired to develop an effective antibiotic delivery system that can cross the cell membrane without compromising its cytocompatibility. Nanoparticles (NPs) have been described as candidates for drug delivery owing to their ability to sometimes penetrate tissues and undergo cellular internalization. A review by Wang et al. highlights nanoparticles used so far to deliver antibiotics intracellularly (Biomaterials Science, 2023, DOI: 10.1039/D2BM01489K). However, the use of antibiotics, while effective in combating infections, inevitably comes with the risk of promoting antibiotic resistance. Consequently, there is growing interest in exploring alternative or adjuvant strategies to traditional antibiotics. An example of for instance metal ions combined with an antibiotic that together exert strong effects is zinc in combination with vancomycin (Zarkan, A., et al., Scientific Reports, 2017. 7(1): p. 4893). However, there is no known combination that is known to be useful against intracellular bacteria. There is no known material useful for delivery of such combinations. There is a need for materials that allow localized delivery of antibiotics. There is a need for materials that allow intracellular delivery of antibiotics. There is a need for materials that allow localized co-delivery of different substances. There is a need for treatment of intracellular infections.

### SUMMARY OF THE INVENTION

The inventors have identified a method for the provision of spherical hydroxyapatite nanoparticles. Use of these particles in the production of colloidal composite gels surprisingly yielded gels with improved properties, such as improved robustness to destructive shear. These gels can be considered to have improved self-healing properties. Active ingredients can be released from the gel in an attractive manner, achieving effective combined release to achieve for instance intracellular antibiotic activity.

Accordingly, the invention provides a method for producing spherical hydroxyapatite nanoparticles, the method comprising the steps of:
i) providing an aqueous calcium salt composition;
ii) providing an aqueous phosphate composition;
iii) adding the aqueous calcium salt composition to the aqueous phosphate composition to obtain a reaction mixture wherein spherical hydroxyapatite (sHA) nanoparticles are formed; and optionally
iv) purifying the sHA nanoparticles.

Preferably the aqueous calcium salt composition is an aqueous solution or suspension comprising 30 to 150 mM calcium salt, preferably 50 to 110 mM, more preferably 75 to 90 mM such as about 83.5 mM calcium salt. Preferably the calcium salt is a hydroxide, a halide, a nitrate, or a salt of an organic acid, preferably a formate, acetate, propionate, malate, lactate, citrate, or gluconate, more preferably a formate, acetate, or propionate, most preferably an acetate.

Preferably the aqueous phosphate composition is an aqueous solution or suspension comprising 20 to 100 mM phosphate, preferably 40 to 60 mM, more preferably 45 to 55 mM such as about 50 mM phosphate, or wherein the phosphate composition comprises about 40-80 mol %, preferably about 50-70 mol %, more preferably about 58-62 such as about 60 mol % phosphate relative to the amount of calcium in the calcium salt composition. Preferably the phosphate is phosphoric acid, a dihydrogen phosphate salt, a hydrogen phosphate salt, or a phosphate salt, preferably a phosphate salt, wherein the salt is preferably a salt of an alkali metal, more preferably of sodium or potassium, most preferably it is a sodium salt.

In preferred embodiments is provided the method, wherein the reaction mixture is allowed to react for at least 2 hours, preferably at least 10 hours, and/or wherein the reaction mixture is at a temperature of about 30 to 50° C., preferably of about 35 to 45 such as about 40° C., and/or wherein sodium citrate is added to the reaction mixture after about 2 hours of reacting.

Preferably the spherical hydroxyapatite nanoparticles are purified by one or more of centrifugation, filtration, decantation, or resuspension, preferably at least by centrifugation and/or resuspension. Preferably the spherical hydroxyapatite nanoparticles have an average size of about 60-500 nm, preferably of about 100-350 nm, more preferably of about 150-250 nm, most preferably about 170-220 nm, or wherein the zeta potential of the spherical hydroxyapatite nanoparticles is in the range of −30 to −15 mV, preferably −18 to −27 mV. Preferably the spherical hydroxyapatite nanoparticles are crystalline.

Provided is a composition comprising spherical hydroxyapatite nanoparticles, preferably as obtainable by a method described above, wherein the composition is preferably an aqueous suspension comprising 10-100 mg/mL of the nanoparticles, more preferably 15-80, even more preferably 20-60, most preferably 25-45 such as about 30 mg/mL.

Provided is the use of spherical hydroxyapatite nanoparticles in the production of a colloidal composite gel, wherein the colloidal composite gel further preferably comprises organic nanoparticles, more preferably gelatine nanoparticles. The nanoparticles used are preferably as described above.

Also provided is a colloidal composite gel comprising
i) organic nanoparticles, more preferably cationic gelatine nanoparticles; and
ii) spherical hydroxyapatite nanoparticles, preferably as obtainable by a method according to the invention.

In this gel, preferably the organic nanoparticles and the spherical hydroxyapatite nanoparticles are present in a weight ratio of 1:10 to 10:1, respectively, or wherein the gel has a total solid content of about 1-50 wt. %, preferably about 10-20 wt. %, more preferably about 15-20 wt. %. Preferably the storage modulus (G′) of the gel is above 1 kPa, preferably wherein the storage modulus of the gel is above 1 kPa after 1, 2, 3, 4, or 5 cycles of destructive shearing.

Also provided is a kit of parts comprising a composition comprising spherical hydroxyapatite nanoparticles according to the invention, and a composition comprising organic nanoparticles as defined above.

The inventors created a resilient gel that could be advantageously used for the killing of intracellular bacteria. The gel had good self-healing properties and was demonstrated as successful at killing intracellular bacteria in a cultured model of intracellular Staphylococcus aureus. The invention provides a colloidal composite gel comprising
i) gelatin nanoparticles; and
ii) spherical hydroxyapatite (sHA) nanoparticles,
   wherein at least one of the gelatin nanoparticles and the sHA nanoparticles comprises an antibiotic agent. Preferably the gelatin nanoparticles comprise a glycopeptide antibiotic. Preferably the gelatin nanoparticles comprise gelatin with an isoelectric point of about 4.7-5.5. Preferably the sHA nanoparticles comprise an antibiotic metal ion. Preferably the sHA nanoparticles comprise at least 12 mol % of the antibiotic metal ion, preferably at least 14 mol %, more preferably at least 15 mol %. Preferably the gelatin nanoparticles comprise vancomycin and wherein the sHA nanoparticles comprise zinc. More preferably the gelatin nanoparticles comprise gelatin with an isoelectric point of about 4.7-5.5 and comprise vancomycin, and wherein the sHA nanoparticles comprise 12-20 mol % zinc. In preferred embodiments the gelatin nanoparticles and the sHA nanoparticles are present in a weight ratio of 1:10 to 10:1, respectively, or wherein the gel has a total solid content of about 5-50 wt. %, preferably about 10-20 wt. %, more preferably about 15-20 wt. %.

Also provided is the colloidal composite gel for use as a medicament. Preferably the medicament is for treatment of intracellular bacterial infection. Preferably the gel kills over 60% of intracellular bacteria within 24 hours after administration. Preferably the gel is administered via injection or via smearing. Preferably the gel is injected or smeared near a joint prosthesis or orthopedic fixation device. The gel can be used in a method of treating an intracellular bacterial infection, the method comprising the step of administering a composite colloidal gel to a subject.

Also provided is a kit of parts comprising a first composition comprising sHA nanoparticles as defined above, and a second composition comprising gelatin nanoparticles as defined above.

### DESCRIPTION OF EMBODIMENTS

The inventors have identified a method for the provision of spherical hydroxyapatite nanoparticles, which can be used to manufacture colloidal composite gels which have improved mechanical properties. Provided is a method for producing spherical hydroxyapatite nanoparticles, the method comprising the steps of:
i) providing an aqueous calcium salt composition;
ii) providing an aqueous phosphate composition;
iii) adding the aqueous calcium salt composition to the aqueous phosphate composition to obtain a reaction mixture wherein spherical hydroxyapatite (sHA) nanoparticles are formed; and optionally
iv) purifying the sHA nanoparticles.

Such a method is referred to herein as a method according to the invention. In preferred embodiments step iv) in not optional. In preferred embodiments the steps of the method are performed in an order wherein step iv) is performed after step iii), and step iii) is performed after steps i) and ii).

The inventors have also identified a method for the provision of resilient gels loaded with antibiotic agents. The gels comprise spherical hydroxyapatite nanoparticles and gelatin nanoparticles, either or both of which can comprise an antibiotic agent. The particles can be used to manufacture colloidal composite gels which have improved mechanical properties. Thus the invention also provides a colloidal composite gel comprising
i) gelatin nanoparticles; and
ii) spherical hydroxyapatite (sHA) nanoparticles,
wherein at least one of the gelatin nanoparticles and the sHA nanoparticles comprises an antibiotic agent. In some embodiments the sHA nanoparticles comprise an antibiotic agent. In some embodiments the gelatin nanoparticles comprise an antibiotic agent. In some embodiments the sHA nanoparticles comprise an antibiotic agent and the gelatin nanoparticles comprise an antibiotic agent. In some embodiments the gel comprises a single antibiotic agent. In some embodiments the gel comprises two different antibiotic agents. In some embodiments the gel comprises more than two different antibiotic agents. Preferably each type of particle comprises at most one type of antibiotic agent, and accordingly it is preferred that the gel comprises one or two different antibiotic agents.

### Nanoparticles

Nanoparticles of the invention can be spherical hydroxyapatite (HA) nanoparticles. Hydroxyapatite is also referred to as hydroxylapatite and is a naturally occurring mineral form of calcium apatite with the formula Ca₅(PO₄)₃(OH). It can also be represented as Ca₁₀(PO₄)₆(OH)₂ to denote that the crystal unit cell comprises two entities. In HA the OH⁻ ion can be replaced by fluoride, chloride, or carbonate, producing fluorapatite or chlorapatite, although the OH⁻ ion is preferred. HA crystallizes in the hexagonal crystal system. In preferred embodiments, the HA nanoparticles comprise an antibiotic agent, preferably an antibiotic metal ion.

As understood herein, nanoparticles are particles with dimensions that generally do not exceed 500 μm in size along their longest dimension, preferably they do not exceed 100 μm in that dimension. Nanoparticles according to the invention are spherical, which a skilled person understands to in this context not require mathematically perfect spherical shapes. In the context of the invention, spherical nanoparticles are generally isotropic but can be oval, egg-shaped, pear-shaped, or have other imperfections as compared to a perfect sphere. In preferred embodiments the spherical nanoparticles are smooth, in that for instance they do not have edges, ridges, or corners. In a perfect sphere, each dimension is of the same length because the sphere is perfectly symmetrical. In preferred embodiments of the nanoparticles of the invention, the shortest dimension of the spherical nanoparticles is within 60% of the longest dimension, more preferably within 70%, still more preferably within 80%, still more preferably within 90%, even more preferably within 95%. Size of dimensions of the nanoparticles can be measured by known methods, for example, by using transmission electron microscopy (TEM) or light scattering techniques, preferably as described in the examples.

Polydispersity of nanoparticles is preferably from 0 to 0.4, more preferably from 0 to 0.35, more preferably from 0 to 0.3, more preferably from 0.05 to 0.28, more preferably from 0.1 to 0.25, more preferably from 0.12 to 0.23, more preferably from 0.14 to 0.22, more preferably from 0.16 to 0.21, more preferably from 0.17 to 0.2, more preferably from 0.18 to 0.2, such as 0.19. In preferred embodiments of the method according to the invention, the polydispersity of the spherical hydroxyapatite nanoparticles is as described above.

In preferred embodiments of the method according to the invention, the spherical hydroxyapatite nanoparticles have an average size of about 60-500 nm, preferably of about 100-350 nm, more preferably of about 150-250 nm, most preferably about 170-220 nm, or the zeta potential of the spherical hydroxyapatite nanoparticles is in the range of −30 to −15 mV, preferably −18 to −27 mV. A preferred size for nanoparticles is about 60-500 nm, preferably 70-450, more preferably 80-400, more preferably 90-375, more preferably 100-325, more preferably 110-300, more preferably 120-275, more preferably 130-230, more preferably 140-225. A preferred zeta potential of the spherical hydroxyapatite nanoparticles is in the range of −30 to −10 mV, preferably −28 to −11 mV, preferably −26 to −12 mV, preferably −26 to −13 mV, preferably −24 to −14 mV, preferably −23 to −16 mV, preferably −22 to −17 mV, preferably −21 to −18 mV, or −21 to −19 mV. sHA nanoparticles of any size can be suitable for use in a gel as described herein.

Size of a nanoparticle can be determined using known techniques such as scanning electron microscopy or light scattering. In preferred embodiments the size is considered to be the hydrodynamic diameter of nanoparticles dispersed in deionized water characterized using dynamic light scattering, for instance using a Zetasizer Lab apparatus (Malvern Instruments Ltd.). Zeta-potential is preferably determined using the same instrument and using nanoparticles dispersed in 5 mM HEPES buffer at pH 7.4.

The molecular and crystal structure of lyophilized spherical CaP nanoparticles and of needle-like CaP nanoparticles were analysed using attenuated total reflectance Fourier transform infrared spectroscopy (ATR-FTIR, Perkin Elmer) and X-ray diffraction (XRD, Phillips X'Pert, PW3710), respectively. The morphology of the particles was visualized by scanning electron microscopy (SEM, JEOL 6301). The average size of dried particles was determined by averaging the length and width of at least 200 particles using SEM images by digital image analysis software (Image J, NIH).

In preferred embodiments, the spherical hydroxyapatite nanoparticles are crystalline. As used herein, crystalline particles preferably have a high crystallinity degree, and the expression crystallinity degree is intended to indicate the percentage of the hydroxyapatite compound present in the crystalline state. Crystallinity degree can be measured according to known methods, such as, for example, by using x-ray diffraction analysis as used in the examples. Preferred crystalline hydroxyapatite nanoparticles have diffraction peaks at 2θ=25.9°, 31.9°, 32.9°, 34.1°, 39.9°, 46.6°, and 49.5°. Preferably the hydroxyapatite nanoparticles are phase-pure.

### Step i) Providing an Aqueous Calcium Salt Composition

In step i) an aqueous calcium salt composition is provided. As used herein, aqueous compositions preferably comprise a solvent system that consist of at least 70% water, more preferably 75%, more preferably 80%, more preferably 85%, more preferably 90%, still more preferably 95, 96, 97, 98, or 99% water. In most preferred embodiments the solvent is essentially water. Water is preferably demineralised water.

In preferred embodiments the aqueous calcium salt composition is an aqueous solution or suspension comprising 30 to 150 mM calcium salt, preferably 50 to 110 mM, more preferably 75 to 90 mM such as about 83.5 mM calcium salt. In some embodiments the composition is a suspension. It is most preferred that the composition is a solution. It was found that spherical hydroxyapatite (sHA) nanoparticles could be obtained when calcium salt concentrations ranging from 16.7 to 417.5 mM were used. Accordingly preferred calcium salt compositions comprise 10 to 500, 15 to 450, 20 to 400, 25 to 350, 35 to 300, 40 to 250, 45 to 200, or 55 to 150 mM calcium salt.

The calcium salt is preferably a hydroxide, a halide, a nitrate, or a salt of an organic acid, preferably a formate, acetate, propionate, malate, lactate, citrate, or gluconate, more preferably a formate, acetate, or propionate, most preferably an acetate. In preferred embodiments the calcium salt is a salt that is soluble in water, preferably that is soluble in water at the concentration used, more preferably that is soluble in water at 30 mM or above, or at 100 mM or above. For this, it is preferred that the salt is a halide, a nitrate, or a salt of an organic acid. A preferred halide is a chloride. Highly preferred is a salt of an organic acid, with acetate being most preferred.

A skilled person will know how much of the aqueous calcium salt composition is required. In preferred embodiments about 1 to 10,000 mL is provided, more preferably about 10 to 5000 mL, more preferably about 25 to 2500 mL, more preferably about 50 to 1000 mL, more preferably about 60 to 750 mL, or about 70 to 500 mL, or about 80 to 350 mL, or about 90 to 250 mL.

In preferred embodiments the calcium salt composition does not comprise further additives. The pH of the composition is preferably acidic, it can be for instance in the range 0-6, or 0.2-4, or 0.4-3, or 0.6-2, or 0.8-1.5, preferably 0.85-1.3, such as about 1. The temperature of the composition is not important and room temperature is preferred for convenience.

### Step ii) Providing an Aqueous Phosphate Composition

In step ii) an aqueous phosphate salt composition is provided. As used herein, aqueous compositions preferably comprise a solvent system that consist of at least 70% water, more preferably 75%, more preferably 80%, more preferably 85%, more preferably 90%, still more preferably 95, 96, 97, 98, or 99% water. In most preferred embodiments the solvent is essentially water. Water is preferably demineralised water.

In preferred embodiments the aqueous phosphate composition is an aqueous solution or suspension comprising 20 to 100 mM phosphate, preferably 40 to 60 mM, more preferably 45 to 55 mM such as about 50 mM phosphate, or wherein the phosphate composition comprises about 40-80 mol %, preferably about 50-70 mol %, more preferably about 58-62 such as about 60 mol % phosphate relative to the amount of calcium in the calcium salt composition. Herein, mol % refers to the amount of phosphate ions compared to the amount of calcium ions. It is most preferred that the phosphate content is adjusted to the calcium content of the composition of step i). For instance, if calcium content is in range of 16.7 mM to 417.5 mM, then phosphate is preferably in the range of 10 mM to 250 mM. For example when phosphate is 50 mM then calcium is preferably 83.5 mM (Ca/P=1.67). In some embodiments the composition is a suspension. It is most preferred that the composition is a solution.

In preferred embodiments the phosphate is phosphoric acid, a dihydrogen phosphate salt, a hydrogen phosphate salt, or a phosphate salt, preferably a phosphate salt, wherein the salt is preferably a salt of ammonia or of an alkali metal, more preferably of an alkali metal, even more preferably sodium or potassium, most preferably it is a sodium salt. Alkali salts are preferred for lack of associated odour, which can conflict with clinical applications.

A skilled person will know how much of the aqueous phosphate salt composition is required. In preferred embodiments about 1 to 10,000 mL is provided, more preferably about 10 to 5000 mL, more preferably about 25 to 2500 mL, more preferably about 50 to 1000 mL, more preferably about 60 to 750 mL, or about 70 to 500 mL, or about 80 to 350 mL, or about 90 to 250 mL. Preferably equal volumes of phosphate salt composition and of calcium salt composition are provided. Preferably, about 10 equivalents of calcium are provided for each 6 equivalents of phosphate.

In preferred embodiments the phosphate salt composition does not comprise further additives. The pH of the composition is preferably basic, it can be for instance in the range 7-14, or 8-13.5, or 9-13.3, or 10-13.2, or 11-13.1, preferably 12-13, such as about 12.8. The temperature of the composition is not important and room temperature is preferred for convenience.

Step iii) Formation of Nanoparticles

In step iii) of the method according to the invention the sHA are formed. It comprises adding the aqueous calcium salt composition of step i) to the aqueous phosphate composition of step ii) to obtain a reaction mixture wherein sHA are formed. In highly preferred embodiments the calcium salt composition is a solution and is added to the phosphate salt composition that is also a solution.

Generally, when a first composition is added to a second composition, the second composition is initially present in excess. The first composition is gradually contacted with the second composition until the entirety of the first composition has been added, at which point both compositions can fully mix. The identity of which composition was in excess at a given point in time can have an influence on the kinetics of the mixing, and thus on any reactions that occur as a result of the mixing. Preferably, adding the aqueous calcium salt composition of step i) to the aqueous phosphate composition of step ii) to obtain a reaction mixture is not an instantaneous addition of the entire calcium salt composition. More preferably the addition performed over at least 1, 2, 3, 4, 5, 6, 7, 8, 9, or 10 seconds. In preferred embodiments the composition of step i) is dripped or poured into the composition of step ii), preferably over a time period of at least 2, 4, 6, 8, 10, 12, 14, 16, 18, 20, 25, 30, 35, 40, 45, 50, 55, or 60 seconds, or more.

The addition can be performed in any way known to a skilled person, such as dripping, pouring, or other forms of admixing. Pouring is convenient at bench scales. The addition is preferably fast, in that there is no addition over a prolonged amount of time. As such, the addition is preferably via direct pouring or fast dripping or injecting to complete addition within at most 5, 4, 3, 2, or 1 minutes. Generally, a composition to which another composition is added is initially present in excess, with the other composition gradually becoming present and mixing into the newly formed mixture.

The addition is preferably performed under strong mixing conditions such as under stirring, preferably vigorous stirring. The addition can be considered a wet-chemical precipitation. It follows the following reaction scheme:

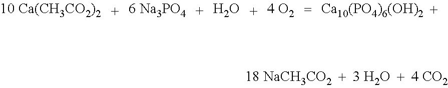

The addition starts the reacting to form hydroxyapatite, in this case sHA nanoparticles. Preferably the reaction mixture is allowed to react for at least 2 hours, preferably at least 10 hours, and/or wherein the reaction mixture is at a temperature of about 30 to 50° C., preferably of about 35 to 45 such as about 40° C., and/or wherein sodium citrate is added to the reaction mixture after about 2 hours of reacting. The reaction mixture is preferably allowed to react for at least 3, 4, 5, 6, 7, 8, 9, 10, 11, 12, 13, 14, 15, 16, 17, or 18 hours, such as 18 hours. The mixture can also be allowed to react for 20, 24, 28, 32, or 36 hours or more. The reaction mixture is preferably allowed to react at a temperature of at least about 31, 32, 33, 34, 35, 36, 37, 38, 39, or 40° C., and of at most about 49, 48, 47, 46, 45, 44, 43, 42, 41, or 40° C. Good results were obtained at about 35-45° C., such as at 40° C.

The reaction mixture preferably has a basic pH, more preferably a pH of about 8-11, more preferably about 8.2-10, more preferably about 8.5-9.5 such as about 9. Preferably the pH of the reaction mixture is not adjusted after addition of the calcium composition to the phosphate composition. This simplifies the experimental procedure, and can be conveniently achieved when the pH of the calcium salt composition is about 1, and the pH of the phosphate composition is about 12.8. A skilled person knows how to calculate pH values and how to predict changes in pH after mixture of compositions.

In preferred embodiments step iii) further comprises the addition of a surface charge modifier to the reaction mixture. This is preferably done after some particles have already formed, for instance after about 4, 6, 8, 10, 12, 14, 16, 18, or 20 hours, most preferably after about 18 hours. Suitable agents for modifying surface charge are known in the art, such as amino acids, small organic acids such as citric acid or EDTA, small organic bases such as lysine, arginine, or spermine, and polymeric acids or bases such as polylysine or polyacrylic acid. Salts of acids or bases can be used. For organic acids, it is preferred that sodium salts are used.

For formation of sHA nanoparticles comprising an antibiotic agent, the agent is preferably added to the phosphate solution, optionally prior to addition of the calcium salt. For formation of zinc-containing sHA nanoparticles, the addition of aqueous zinc salt such as zinc nitrate prior to the addition of calcium salt can lead to good results. The zin salt can be 50-200 mM, preferably 75-150 mM, such as preferably 100 mM. The amount of zinc depends on the desired molar percentage. For other antibiotic metal ions, different salts can be used as appropriate.

Good results were obtained when the surface charge modifier was to render the surfaces of the sHA nanoparticles negatively charged, for instance using organic acids, particularly citric acid, more preferably citrate such as sodium citrate. Addition of the surface charge modifiers was found to render the particles more stable, preventing clustering.

After addition of a surface charge modifier, the reaction mixture is preferably allowed to react for an additional amount of time, for instance 0.5, 1, 2, 3, 4, 5, 6, 7, 8, 9, 10, 11, 12, 13, 14, 15, 16, 17, or 18 hours more, or more. Addition of the surface charge modifier can be via controlled addition over a an amount of time, such as over 10-300 minutes, or 30-240 minutes, or 60-180 minutes, or 90-150 minutes, such as 120 minutes. Good results were obtained when the modifier such as citrate was added over 120 minutes.

The surface charge modifier can be added to the reaction mixture in any way. For instance, a dry powder can be added to the reaction mixture, particularly while the reaction mixture is stirred or vigorously stirred. The surface charge modifier is preferably used in an amount relative to the amount of calcium. Preferably, about 0.05 to 0.5 equivalents are used, more preferably about 0.1 to 0.4, more preferably 0.15 to 0.3, most preferably about 0.17 to 0.25, such as about 0.21 equivalents. For example, when 83.5 mM calcium is present, about 18.2 mM sodium citrate can be used.

### Step iv) Purifying the Nanoparticles

The sHA nanoparticles can be purified for further use. In preferred embodiments the method according to the invention further comprises step iv) purifying the nanoparticles. Several known methods exist for purifying small inorganic nanoparticles such as HA nanoparticles, and these methods are also suitable for the sHA nanoparticles of the invention.

In preferred embodiments the spherical hydroxyapatite nanoparticles are purified by one or more of centrifugation, filtration, decantation, or resuspension, preferably at least by centrifugation and/or resuspension. Centrifugation is preferably at about 5,000 to 15,000 G, such as at about 11,000 to 12,000 G, for instance 11,648 G. This is for instance 10,000 rpm, using a Heraeus Multifuge X1R, Thermo Fisher. Centrifugation is preferably performed for about 1 to 30 minutes, more preferably about 2 to 25, 3 to 20, 4 to 15, 5 to 14, 6 to 13, 7 to 12, 8 to 11, or 9 to 10 minutes such as about 10 minutes. After centrifugation the supernatant is preferably discarded.

Resuspension is preferably in water such as in deionized water, and resuspension can be repeated 2, 3, 4, or 5 times or more. In preferred embodiments sHA nanoparticles are resuspended at least once, preferably three times, more preferably after centrifugation.

### Use of the Nanoparticles

It was found that sHA nanoparticles according to the invention imparted attractive properties on gels. Accordingly the invention provides the use of spherical hydroxyapatite nanoparticles in the production of a colloidal composite gel, wherein the colloidal composite gel further preferably comprises organic nanoparticles, more preferably gelatine nanoparticles. The sHA nanoparticles are as described above. The invention also provides a method for preparing a colloidal composite gel, the method comprising the mixing of sHA nanoparticles as described above with gelatine nanoparticles as described herein. Preferably the gel comprises only one type of sHA nanoparticles and only one type of organic nanoparticles.

Colloidal composite gels are known as such, and are for instance described by Wang et al. (Acta Biomaterialia, 2013, doi: 10.1016/j.actbio.2013.08.036). A composite gel comprises both organic and inorganic nanoparticles. A colloidal gel is generally formed or stabilized via attractive interparticle interactions. Suitable such interactions are Vanderwaals forces, hydrophobic interactions, electrostatic interactions, and host-guest interactions. For instance colloidal gels can comprise both cationic organic nanoparticles and anionic inorganic nanoparticles.

Organic nanoparticles can be any type of organic nanoparticles known in the art. Examples are polypeptide nanoparticles, lipid nanoparticles, polysaccharide nanoparticles, and polymer nanoparticles. Preferred polypeptide nanoparticles are based on natural polymers or lipids, such as lipids, silk, dextran, gelatine, collagen, alginate, gellan gum, carrageenan, chitosan, fibrin, or polypeptide nanoparticles. More preferably nanoparticles are based on structural proteins or materials derived thereof, such as collagen or gelatine. Gelatine nanoparticles (GNPs) are most preferred. GNPs are known in the art and can for instance be provided as described in the examples. Nanoparticles can be of gelatine type A or of gelatine type B. In some embodiments, preferred gelatine is gelatine type A. In some embodiments, preferred gelatin is gelatin type B. In some embodiments, the gelatin preferably has an isoelectric point of about 4.7-5.5. The gel according to the invention preferably comprises gelatin nanoparticles that comprise gelatin with an isoelectric point of about 4.7-5.5. GNPs are preferably crosslinked, more preferably using carbodiimides such as EDC or DCC. GNPs can be conveniently handled and stored in an aqueous suspension, such as a suspension of 30 mg mL⁻¹ in demineralized water, preferably at 4° C. Organic nanoparticles are preferably gelatin nanoparticles. Organic nanoparticles can be cationic or anionic nanoparticles. In some embodiments organic nanoparticles are anionic nanoparticles. In some embodiments organic nanoparticles are cationic nanoparticles. Organic nanoparticles are preferably spherical nanoparticles.

GNPs can be prepared by a desolvation process, for instance using ethanol. Generally, gelatin (such as 1.25 g gelatin type A or type B) can be dissolved in demineralized water (such as 25 mL) preferably under constant stirring (500 rpm), for instance at 40° C. The pH can then be lowered to for example 3.0 using for example 1 M HCl whereafter ethanol (such as about 100 mL) can be added, preferably dropwise and preferably under constant stirring. This induces gelatin desolvation and aggregation into spherical Gel NPs. The Gel NPs can be crosslinked, for example by the addition of 5 mL of 5 mM, 1-ethyl-3-(3-dimethyl-aminopropyl)carbodiimide and preferably 1 mM N-hydroxysuccinimide dissolved in deionized water, preferably under constant stirring (500 rpm), preferably at room temperature. Washing can be done using filters, for instance with a 300 kDa cutoff membrane. Glycopeptide antibiotics can be loaded into the GNPs. For example to produce vancomycin-loaded GNPs (VGNPs), 100 μg vancomycin (Sigma-Aldrich) can be dissolved in 1 mL of a suspension of GNPs in demineralized water (8 mg/mL) and equilibrated at for example 4° C. for at least 12 h. This allows absorption and diffusion of vancomycin to the Gel NPs. Afterwards, the VGNPs can be centrifuged for example at 10.000×g for 10 min to remove unbound vancomycin, after which particles can be resuspended in phosphate buffered saline (PBS). Other antibiotic agents can be used as desired.

Properties of GNPs can be determined using known methods, preferably using a dynamic light scattering device, more preferably as described in the examples. At pH 7.4, preferred GNPs have a hydrodynamic diameter of 50 to 100,000, preferably 100 to 50,000, more preferably 150 to 10,000 more preferably 200 to 1,000 nm, even more preferably 210 to 750 nm, still more preferably 220 to 500 nm, most preferably 230 to 400 or 230 to 300 or 230 to 250 nm, such as 237.1±2.3 nm; with a polydispersity index of 0.00 to 0.07, more preferably 0.02 to 0.06, more preferably 0.03 to 0.05 such as 0.04±0.02; and a zeta-potential of +5 to +30, preferably +10 to +25, more preferably of +15 to +22 such as +18.8±0.6 mV.

The uses preferably encompasses mixing of an aqueous suspension of organic nanoparticles such as GNPs with an aqueous suspension of sHA nanoparticles. Generally, the nanoparticles will coagulate after mixing, often immediately. It is preferred that after mixing the dispersions are allowed to equilibrate for about 10-240, preferably 20-210, more preferably 30-180, preferably 40-150, more preferably 50-120, most preferably 55-90 minutes such as 60 minutes. The colloidal composite gels can be freeze-dried after formation, for instance for 48 h. Colloidal gels can be prepared at different total solid content, for instance in the range of 1-50 or 2-40 wt %, preferably 5-35 wt %, more preferably 7-30 wt % most preferably 10-20 wt % such as 10 wt %, 15 wt %, or 20 wt %. The weight ratio of sHA nanoparticles to organic nanoparticles can be for example in the range of 0.5 to 5, or 0.6 to 4, or 0.7 to 3, or 0.8 to 3.5, or 0.9 to 3, or 1 to 2, such as sHA-NPs/GNPs weight ratios of 1 or 2, respectively. Gels can also be prepared in aqueous buffers or in other clinically relevant media such as in cell culture medium, for instance DMEM or EMEM or PBS or HBS or alpha-MEM. It was found that the gels could form independent of the ionic strength of the liquid medium used.

### Antibiotic Agents

Antibiotic agents are widely known, and can treat or prevent bacterial infections or bacterial proliferation. Particularly suitable antibiotic agents are small molecules and antibiotic metal ions. A small molecule preferably has a molecular weight of at most 1000 Da, preferably at most 600 Da. Suitable antibiotic metal ions can be selected by a skilled person, and examples are zinc, gold, silver, copper, gallium, manganese, platinum, and palladium.

Preferably, when the sHA nanoparticles comprise an antibiotic agent, they comprise an antibiotic metal ion. In preferred embodiments the gel is provided, wherein the sHA nanoparticles comprise an antibiotic metal ion. This antibiotic metal ion is preferably selected from zinc, gold, silver, copper, gallium, manganese, platinum, and palladium, more preferably from zinc and silver, most preferably it is zinc. Metal ions are attractive because it was found that at concentrations below about 25 mol % the metal, such as zinc, was incorporated in the crystal structure of the hydroxyapatite. Therefore in preferred embodiments the sHA nanoparticles comprise at most 25, preferably at most 22, more preferably at most 20 mol % antibiotic agent such as an antibiotic metal ion. Preferably the sHA nanoparticles comprise at least 0.5, more preferably 1, still more preferably 5, even more preferably 10, even more preferably 12 mol % antibiotic agent such as an antibiotic metal ion. In preferred embodiments the sHA nanoparticles comprise at least 12 mol % of the antibiotic metal ion, preferably at least 14 mol %, more preferably at least 15 mol %. Accordingly, the sHA nanoparticles preferably comprise 12-20, more preferably 14-20, most preferably 15-20 mol %.

Preferably, when the gelatin nanoparticles comprise an antibiotic agent, they comprise a glycopeptide antibiotic. In preferred embodiments the gel is provided, wherein the gelatin nanoparticles comprise a glycopeptide antibiotic. Glycopeptide antibiotics are a class of drugs generally of microbial origin that are composed of glycosylated cyclic or polycyclic nonribosomal peptides. Significant glycopeptides include the antibiotics vancomycin, teicoplanin, telavancin, ramoplanin, decaplanin, corbomycin, complestatin, and bleomycin. Vancomycin can be used if infection with methicillin-resistant bacteria such as Staphylococcus aureus (MRSA) is suspected, which makes vancomycin a preferred antibiotic agent, particularly for gels as described herein. In preferred embodiments the gelatin nanoparticles comprise at most 200, preferably at most 150, more preferably at most 100 μg antibiotic, preferably glycopeptide antibiotic, most preferably vancomycin per mL of gelatin nanoparticle suspension. In preferred embodiments the gelatin nanoparticles comprise at most 200, preferably at most 150, more preferably at most 100 μg antibiotic, preferably glycopeptide antibiotic, most preferably vancomycin per 8 mg of gelatin nanoparticles. Nanoparticles preferably comprise at least 1, preferably 5, more preferably 10, still more preferably 50 μg glycopeptide antibiotic per mL suspension. Nanoparticles preferably comprise at least 1, preferably 5, more preferably 10, still more preferably 50 μg glycopeptide antibiotic per mg nanoparticles.

In preferred embodiments the gel comprises gelatin nanoparticles that comprise glycopeptide antibiotic, more preferably vancomycin. In preferred embodiments the gel comprises sHA nanoparticles that comprise an antibiotic metal ion, more preferably zinc. It was found that antibiotic metal ions and glycopeptide antibiotics were effective in combination. Accordingly, in highly preferred embodiments the gel comprises gelatin nanoparticles that comprise glycopeptide antibiotic, and sHA nanoparticles that comprise an antibiotic metal ion. Most preferably the gel comprises gelatin nanoparticles that comprise vancomycin and sHA nanoparticles that comprise zinc.

### Products and Compositions

The invention provides a composition comprising spherical hydroxyapatite nanoparticles, preferably as obtainable by a method according to the invention, wherein the composition is preferably an aqueous suspension comprising 10-100 mg/mL of the nanoparticles, more preferably 15-80, even more preferably 20-60, most preferably 25-45 such as about 30 mg/mL. The w/v % of the sHA nanoparticles is not of high importance because various stock solutions can be provided depending on the intended use. For instance suspensions can also comprise 1-500 mg/mL or even less than 1 mg/mL, or more than 500 mg/mL. For practical applications such as gel formation a range of 5-30 w/v % is preferred, more specifically 10-20 w/v %. In preferred embodiments the composition is an aqueous composition, more preferably the composition consists essentially of sHA nanoparticles and water. The sHA nanoparticles comprised in such compositions preferably comprise an antibiotic agent, more preferably an antibiotic metal ion such as zinc.

The composition can also be a paste comprising sHA nanoparticles according to the invention, wherein the paste preferably comprises about 25 to 50 w/v % sHA nanoparticles, more preferably about 30-40 w/v %. The paste is preferably water-based, and more preferably the paste consists essentially of water and sHA nanoparticles.

The invention also provides a colloidal composite gel comprising
i) organic nanoparticles, more preferably cationic gelatine nanoparticles; and
ii) spherical hydroxyapatite nanoparticles, preferably as obtainable by a method according to the invention.

The invention also provides a colloidal composite gel comprising
i) organic nanoparticles, more preferably gelatin nanoparticles such as cationic gelatin nanoparticles; and
ii) spherical hydroxyapatite nanoparticles, preferably as obtainable by a method according to the invention.
   Such gels are referred to herein as a gel according to the invention. Preferably at least one of the gelatin nanoparticles and the sHA nanoparticles comprises an antibiotic agent. The gels are robust to mechanical attack which can be relevant when the gel is to be used in confined spaces, or when the gel is exposed to mechanical stresses. A parameter that is relevant for these beneficial properties is the storage modulus G′, which is an indication of the gel's ability to store deformation energy in an elastic manner. G′ can be determined using known techniques, for instance as described in the Examples. Preferred gels according to the invention have a storage modulus of at least 100 Pa, which may be useful for certain applications where soft gels are required. Other preferred gels have a storage modulus of at least 500, 1000, 1200, or preferably 1400 Pa. Preferred gels have a storage modulus of at most 200 kPa, preferably at most 100 kPa, more preferably at most 50 kPa, most preferably at most 10 kPa. Preferred gels according to the invention have a storage modulus of about 0.1-200 kPa, preferably about 0.5-150 kPA, more preferably about 1-125 kPa, more preferably about 1.2-100 kPa, even more preferably about 1.4-75 kPa, more preferably still 1.6-50 kPa, or 1.8-40 kPa, or 2-30 kPa, or 2.1-25 kPa, or 2.2-20 kPa, or 2.3-15 kPa, most preferably 2-10 kPa or 2.4-9 kPa.

In preferred embodiments, the organic nanoparticles and the spherical hydroxyapatite nanoparticles are present in a weight ratio of 1:10 to 10:1, respectively, or the gel has a total solid content of about 5-50 wt. %, preferably about 10-20 wt. %, more preferably about 15-20 wt. %. The organic nanoparticles are preferably as described above.

Preferably the organic nanoparticles and the spherical hydroxyapatite nanoparticles are present in a weight ratio of 1:10 to 10:1, or of 1:9 to 9:1, or 1:8 to 8:1, or 1:7 to 7:1, or 1:6 to 6:1, or 1:5 to 5:1, or 1:4 to 4:1, or 1:3 to 3:1, or 1:2 to 2:1, or about 1:1, respectively.

Preferably the gel has a total solid content of about 5-50 wt. %, preferably about 10-20 wt. %, more preferably about 15-20 wt. %. The gels can have a total solid content that is for instance in the range of 2-40 wt %, preferably 5-35 wt %, more preferably 7-30 wt % most preferably 10-20 wt % such as 10 wt %, 15 wt %, or 20 wt %. In preferred embodiments the gel has a total solid content of 5-50, 10-40, 10-20, or most preferably 15-20 wt %. The weight ratio of sHA nanoparticles to organic nanoparticles can be for example in the range of 0.5 to 5, or 0.6 to 4, or 0.7 to 3, or 0.8 to 3.5, or 0.9 to 3, or 1 to 2, such as sHA-NPs/GNPs weight ratios of 1 or 2, respectively. The gels can comprise water or aqueous buffers or other clinically relevant media such as in cell culture medium, for instance DMEM or EMEM or PBS or HBS or alpha-MEM. Preferably the total solid content is for at least 70 wt % composed of the sHA nanoparticles and the organic nanoparticles, more preferably at least 75 wt %, more preferably at least 80 wt %, more preferably at least 85, more preferably at least 90, more preferably at least 95, more preferably at least 96, 97, 98, 99, or 100 wt %.

In preferred embodiments of the gel according to the invention, the storage modulus (G′) of the gel is above 2 kPa, preferably wherein the storage modulus of the gel is above 2 kPa after 1, 2, 3, 4, or 5 cycles of destructive shearing. The storage modulus can remain above 2 kPa after additional cycles. It is preferred that the storage modulus remains within a certain percentage of its original value before repeated destructive shearing. In preferred embodiments the storage modulus of the gel after 5 cycles of destructive shear remains within 50% of the storage modulus after a first cycle of destructive shear, preferably within 60%, more preferably 70%, more preferably 80%, more preferably 90%.

In preferred embodiments of the gel according to the invention, the storage modulus (G′) of the gel is above 2 kPa, preferably wherein the storage modulus of the gel is above 2 kPa after 1, 2, 3, 4, or 5 cycles of extrusion. The storage modulus can remain above 2 kPa after additional cycles. It is preferred that the storage modulus remains within a certain percentage of its original value before repeated extrusion. In preferred embodiments the storage modulus of the gel after 5 cycles of extrusion remains within 50% of the storage modulus after a first extrusion, preferably within 60%, more preferably 70%, more preferably 80%, more preferably 90%. For this, extrusion is preferably through a syringe with a gauge dimension from 7-34 G, preferably 12-26 G, more preferably 16-22 G such as 20 G.

Destructive shear is a common technique. In practice, when G″ is brought above G′ through shear, the shear is destructive. Gels of the invention reform and regain their storage modulus G′. A preferred destructive shear is an oscillatory strain sweep, for instance with increasing strain from 0.1% to 1000%, preferably with a fixed angular frequency, such as of 1 rad/s. Extrusion can be extrusion through a syringe, and multiple cycles can be extrusion through a syringe, back and forth.

Also provided is a kit of parts comprising a composition comprising spherical hydroxyapatite nanoparticles according to the invention, and a composition comprising organic nanoparticles, preferably as defined earlier herein, preferably wherein at least one of the two types of nanoparticles comprises an antibiotic agent. In preferred embodiments a kit of parts is provided, comprising a first composition comprising sHA nanoparticles comprising an antibiotic agent as defined above, and a second composition comprising gelatin nanoparticles comprising an antibiotic agent as defined above. Also provided is a kit of parts comprising a gel according to the invention and a syringe needle, preferably a syringe needle with a gauge dimension from 7-34 G, preferably 12-26 G, more preferably 16-22 G such as 20 G. Such a kit can be useful for administering a gel according to the invention to a confined space in a subject, for instance to provide mechanical support or to provide lubrication. It can also be useful when a gel according to the invention is to be used in a printer, such as a 3D-printer. Instead of a syringe needle, a tapered tip can also be used, preferably having a gauge of 20 G, 22 G or 25 G, more preferably 22 G.

In preferred embodiments the invention provides the gel as described above, wherein the gelatin nanoparticles comprise gelatin with an isoelectric point of about 4.7-5.5 and comprise vancomycin, and wherein the sHA nanoparticles comprise 12-20 mol % zinc.

Preferably the gelatin nanoparticles and the sHA nanoparticles are present in a weight ratio of 1:10 to 10:1, respectively, or preferably the gel has a total solid content of about 5-50 wt. %, preferably about 10-20 wt. %, more preferably about 15-20 wt. %. In some embodiments Preferably the gelatin nanoparticles and the sHA nanoparticles are present in a weight ratio of 1:9 to 9:1, of 8:1 to 1:8, of 7:1 to 1:7, of 6:1 to 1:6, of 5:1 to 1:5, of 4:1 to 1:4, of 3:1 to 1:3, of 2:1 to 1:2, or of about 1:1. In some embodiments the gel has a total solid content of about 5-50 wt. %, preferably about 7-45 wt. %, more preferably about 9-40, more preferably 11-35, still more preferably about 12-30 wt. %, or 14-25 wt. %, more preferably about 15-20 wt. %.

### Medical Applications

The gels comprising an antibiotic agent are particularly suitable for use as a medicament. The gels are suitable for use as an antibiotic, more particularly for use as an antibacterial agent. The gels are preferably for use in treating bacterial infection. In preferred embodiments, the gels are for treatment of intracellular bacterial infection. Treatment is preferably in a subject in need thereof. In preferred embodiments the subject is a non-human subject. In other preferred embodiments the subject is a human subject. Preferably, the subject is a subject in need of treatment or at risk of infection, most preferably in need of treatment. Need of treatment can be need to cure an infection, but it can also be prophylactic treatment. In preferred embodiments, treatment is primary prophylactic treatment for the prevention of disease. The inventors found that the invention is also particularly suited for secondary prophylaxis, so in other preferred embodiments, treatment is secondary prophylactic treatment for the prevention of disease recurrence after earlier treatment. In preferred embodiments, a subject is elderly. An elderly subject is preferably over 50 years of age, more preferably over 60, even more preferably over 65, more preferably still over 70, most preferably over 75. Preferred subjects have undergone surgery or are about to undergo surgery, preferably orthopedic surgery. Preferred subjects have a joint prosthesis or and orthopedic fixation device.

In the context of this invention, treatment of a bacterial infection may refer to preventing, ameliorating, curing, and/or delaying an infection with bacteria. Prevention of an infection may mean that an infection is cured before symptoms of the infection have manifested. Preferably, successful treatment may mean that:
The severity of at least one symptom of the infection has been reduced, and/or
A symptom has not manifested while this manifestation could have been expected, and/or
At least a parameter associated with the infection has been improved.

Preferred symptoms are selected from the group consisting of inflammation, epithelial destruction, redness, edema, haemorrhage, exudate, fatigue, weight loss, malaise, night sweats, fever, chest pain, decreased exercise tolerance, dyspnoea, and dizziness, more preferably selected from inflammation and fever.

A parameter may be a symptom as described above, or it may preferably be selected from the group consisting of prolongation of patient survival, improvement of the quality of life, observed pain relief, amelioration of a comorbid condition, decrease in the number of live bacteria in samples measured by quantitative cultures, decrease in the number of live bacteria in samples measured by semi-quantitative cultures, no live bacteria detectable by culture in samples, improvement of lesions seen on computed tomography scans, increased exercise tolerance, increased walking distance in the 6-minute walk test, and improvement in quality of life reported in quality of life measurement. More preferably a symptom is selected from the group consisting of prolongation of patient survival, or decrease in the number of live bacteria in samples measured by quantitative cultures.

In the context of the invention, treating, preventing, curing and/or delaying an infection is preferably assessed or detected after at least one day, two days, three days, four days, one week, two weeks, three weeks, four weeks, one month, two months, three months, four months, five months, six months or more in a treated subject. Treating, preventing, curing and/or delaying an infection is preferably identified in a subject as:
a prolongation of patient survival of at least one month, several months or more (compared to those not treated or treated with a control or compared with the subject at the onset of the treatment) and/or
improvement of the quality of life and observed symptom relief such as pain relief.

In the context of the invention, a patient may survive and/or may be considered as being disease free. Alternatively, the infection may have been stopped or delayed. In the context of the invention, an improvement of quality of life and observed pain relief may mean that a patient may need less pain relief drugs than at the onset of the treatment. A patient may no longer need any pain relief drug. This improvement of quality of life and observed pain relief may be seen, detected or assessed after at least one week, two weeks, three weeks, four weeks, one month, two months, three months, four months, five months, six months or more of treatment in a patient and compared to the quality of life and observed pain relief at the onset of the treatment of said patient.

Preferably the infection is an opportunistic infection. In preferred embodiments is provided the gel for use according to the invention, wherein the subject is infected with an intracellular infection. Intracellular infection can be any infection, including infections that contribute to tissue damage.

The gels were found to be effective for killing intracellular bacteria. In preferred embodiments the gel kills over 60% of intracellular bacteria within 24 hours after administration. Preferably it kills over 65%, more preferably over 70%, even more preferably over 75%, more preferably over 80%, more preferably over 85%, more preferably over 90%, more preferably over 95%, most preferably it kills 100%. Bacterial kill can be referred to as efficacy and can be determined using techniques known in the art, for example by bacteria count or CFU determination, preferably as described in the examples.

Treatment using gels according to the invention is useful during or after surgery, for instance to prevent post-operatic infection. The gel is therefore preferably applied inside a subject such as in a surgical wound or opening or puncture. In preferred embodiments the gel is administered via injection or via smearing. Injection of the gel benefits from the excellent self-healing properties of the gel. Smearing can be useful when the intended site of application has become accessible during surgery. Preferably, application is on or near a joint prosthesis or orthopedic fixation device. In some embodiments the nanoparticles are applied as dry particles which form a gel in situ after contact with body fluids or with blood. Smearing is preferably smearing onto or into infected tissue, tissue at risk of being infected, or tissue suspected of being infected.

Treatment using the gel for use according to the invention preferably comprises administration of a total of at least 0.1 mg of antibiotic agent, more preferably at least 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 160, 170, 180, 190, or 200 mg per administration, even more preferably at least 10 mg, more preferably still at least 50 mg, even more preferably 60 mg, more preferably 70 mg, more preferably still 80 mg, even more preferably at least 100 mg, more preferably still at least 125 mg, most preferably at least 150 mg per administration. In preferred embodiments the gel is administered only once.

The above use of a product is convenient in a method of treating a bacterial infection, such as a method of treating an intracellular bacterial infection, wherein the method comprises the step of administering a composite colloidal gel as described herein.

### General Definitions

Unless stated otherwise, all technical and scientific terms used herein have the same meaning as customarily and ordinarily understood by a person of ordinary skill in the art to which this invention belongs, and read in view of this disclosure. In this document and in its claims, the verb “to comprise” and its conjugations is used in its nonlimiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention. Gelatin and gelatine can be alternate spellings of the same material.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

Reference to an element by the indefinite article “a” or “an” does not exclude the possibility that more than one of the element is present, unless the context clearly requires that there be one and only one of the elements. The indefinite article “a” or “an” thus usually means “at least one”. As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. The word “about” or “approximately” when used in association with a numerical value (e.g. about 10) preferably means that the value may be the given value (of 10) more or less 10% of the value, or more or less 1% of the value. As used herein, the term “and/or” indicates that one or more of the stated cases may occur, alone or in combination with at least one of the stated cases, up to with all of the stated cases. Various embodiments are described herein. Each embodiment as identified herein may be combined together unless otherwise indicated.

All patent applications, patents, and printed publications cited herein are incorporated herein by reference in their entireties. One skilled in the art will recognize many methods and materials similar or equivalent to those described herein, which could be used in the practice of the present invention. The present invention is in no way limited to the methods and materials described.

### PARTICULAR EMBODIMENTS

1. Method for producing spherical hydroxyapatite nanoparticles, the method comprising the steps of:
   i) providing an aqueous calcium salt composition;
   ii) providing an aqueous phosphate composition;
   iii) adding the aqueous calcium salt composition to the aqueous phosphate composition to obtain a reaction mixture wherein spherical hydroxyapatite (sHA) nanoparticles are formed; and optionally
   iv) purifying the sHA nanoparticles.
2. Method for producing spherical hydroxyapatite nanoparticles having an average size of 60-500 nm, the method comprising the steps of:
   i) providing an aqueous calcium salt composition comprising calcium acetate;
   ii) providing an aqueous phosphate composition;
   iii) combining the aqueous calcium salt composition with the aqueous phosphate composition to obtain a reaction mixture wherein spherical hydroxyapatite (sHA) nanoparticles are formed; and optionally
   iv) purifying the sHA nanoparticles.
3. The method according to embodiment 1 or 2, wherein the aqueous calcium salt composition is an aqueous solution or suspension comprising 30 to 150 mM calcium salt, preferably 50 to 110 mM, more preferably 75 to 90 mM such as about 83.5 mM calcium salt.
4. The method according to any one of embodiments 1-3, wherein the calcium salt is a hydroxide, a halide, a nitrate, or a salt of an organic acid, preferably a formate, acetate, propionate, malate, lactate, citrate, or gluconate, more preferably a formate, acetate, or propionate, most preferably an acetate.
5. The method according to any one of embodiments 1-4, wherein the spherical hydroxyapatite nanoparticles do not comprise organic polymers.
6. The method according to any one of embodiments 1-5, wherein the method does not comprise a calcination step.
7. The method according to any one of embodiments 1-6, wherein the aqueous phosphate composition is an aqueous solution or suspension comprising 20 to 100 mM phosphate, preferably 40 to 60 mM, more preferably 45 to 55 mM such as about 50 mM phosphate, or wherein the phosphate composition comprises about 40-80 mol %, preferably about 50-70 mol %, more preferably about 58-62 such as about 60 mol % phosphate relative to the amount of calcium in the calcium salt composition.
8. The method according to any one of embodiments 1-7, wherein the phosphate is phosphoric acid, a dihydrogen phosphate salt, a hydrogen phosphate salt, or a phosphate salt, preferably a phosphate salt, wherein the salt is preferably a salt of an alkali metal, more preferably of sodium or potassium, most preferably it is a sodium salt.
9. The method according to any one of embodiments 1-8, wherein the reaction mixture is allowed to react for at least 2 hours, preferably at least 10 hours, and/or wherein the reaction mixture is at a temperature of about 30 to 50° C., preferably of about 35 to 45 such as about 40° C., and/or wherein sodium citrate is added to the reaction mixture after about 2 hours of reacting.
10. The method according to any one of embodiments 1-9, wherein the spherical hydroxyapatite nanoparticles are purified by one or more of centrifugation, filtration, decantation, or resuspension, preferably at least by centrifugation and/or resuspension.
11. The method according to any one of embodiments 1-10, wherein the spherical hydroxyapatite nanoparticles have an average size of about 60-500 nm, preferably of about 100-350 nm, more preferably of about 150-250 nm, most preferably about 170-220 nm, or wherein the zeta potential of the spherical hydroxyapatite nanoparticles is in the range of −30 to −15 mV, preferably −18 to −27 mV.
12. The method according to any one of embodiments 1-10, wherein the spherical hydroxyapatite nanoparticles have an average size of about 100-350 nm, preferably of about 150-250 nm, more preferably about 170-220 nm, or wherein the zeta potential of the spherical hydroxyapatite nanoparticles is in the range of −30 to −15 mV, preferably −18 to −27 mV.
13. The method according to any one of embodiments 1-12, wherein the spherical hydroxyapatite nanoparticles are crystalline.
14. Composition comprising spherical hydroxyapatite nanoparticles, preferably as obtainable by a method according to any one of embodiments 1-13.
15. Composition according to embodiment 14, wherein the composition is an aqueous suspension comprising 10-100 mg/mL of the nanoparticles, preferably 15-80, more preferably 20-60, most preferably 25-45 such as about 30 mg/mL.
16. Use of spherical hydroxyapatite nanoparticles in the production of a colloidal composite gel, wherein the colloidal composite gel further preferably comprises organic nanoparticles, more preferably gelatine nanoparticles.
17. Colloidal composite gel comprising
   i) organic nanoparticles, more preferably cationic gelatine nanoparticles; and
   ii) spherical hydroxyapatite nanoparticles, preferably as obtainable by a method according to any one of embodiments 1-13.
18. Gel according to embodiment 17, wherein the organic nanoparticles and the spherical hydroxyapatite nanoparticles are present in a weight ratio of 1:10 to 10:1, respectively, or wherein the gel has a total solid content of about 5-50 wt. %, preferably about 10-20 wt. %, more preferably about 15-20 wt. %.
19. Gel according to embodiment 17 or 18, wherein the storage modulus (G′) of the gel is above 2 kPa, preferably wherein the storage modulus of the gel is above 2 kPa after 1, 2, 3, 4, or 5 cycles of destructive shearing.
20 Kit of parts comprising a composition comprising spherical hydroxyapatite nanoparticles according to embodiment 15, and a composition comprising organic nanoparticles as defined in embodiment 16.
21. Colloidal composite gel comprising
   i) gelatin nanoparticles; and
   ii) spherical hydroxyapatite (sHA) nanoparticles,
wherein at least one of the gelatin nanoparticles and the sHA nanoparticles comprises an antibiotic agent.
22. The gel according to embodiment 21, wherein the gelatin nanoparticles comprise a glycopeptide antibiotic.
23. The gel according to embodiment 21 or 22, wherein the gelatin nanoparticles comprise gelatin with an isoelectric point of about 4.7-5.5.
24. The gel according to any one of embodiments 21-23, wherein the sHA nanoparticles comprise an antibiotic metal ion.
25. The gel according to any one of embodiments 21-24, wherein the sHA nanoparticles comprise at least 12 mol % of the antibiotic metal ion, preferably at least 14 mol %, more preferably at least 15 mol %.
26. The gel according to any one of embodiments 21-25, wherein the gelatin nanoparticles comprise vancomycin and wherein the sHA nanoparticles comprise zinc.
27. The gel according to any one of embodiments 21-26, wherein the gelatin nanoparticles comprise gelatin with an isoelectric point of about 4.7-5.5 and comprise vancomycin, and wherein the sHA nanoparticles comprise 12-20 mol % zinc.
28. The gel according to any one of embodiment 21-27, wherein the gelatin nanoparticles and the sHA nanoparticles are present in a weight ratio of 1:10 to 10:1, respectively, or wherein the gel has a total solid content of about 5-50 wt. %, preferably about 10-20 wt. %, more preferably about 15-20 wt. %.
29. Colloidal composite gel according to any one of embodiments 21-28, for use as a medicament.
30. The gel for use according to embodiment 29, for treatment of intracellular bacterial infection.
31. The gel for use according to embodiment 30, wherein the gel kills over 60% of intracellular bacteria within 24 hours after administration.
32. The gel for use according to any one of embodiments 29-31, wherein the gel is administered via injection or via smearing, preferably via smearing into infected tissues.
33. The gel for use according to embodiment 32, wherein the gel is injected or smeared near a joint prosthesis or orthopedic fixation device.
34. Kit of parts comprising a first composition comprising sHA nanoparticles as defined in any one of embodiments 21-28, and a second composition comprising gelatin nanoparticles as defined in any one of embodiments 21-28.
35. Method of treating an intracellular bacterial infection, the method comprising the step of administering a composite colloidal gel according to any one of embodiments 21-28.

### FIGURE LEGENDS

FIG. 1A—Scanning electron micrograph of spherical HA-NPs. scale bar=200 nm, dispersed prior to imaging in deionized water at a concentration of 0.01% w/v.

FIG. 1B—as for FIG. 1A, but for needle CaP nanoparticles.

FIG. 2—XRD patterns of spherical and needle-shaped HA-NPs. The peaks reflect crystalline material.

FIG. 3—FTIR spectra of spherical-shaped HA-NPs and needle-shaped CaP nanoparticles (denoted nHA-NPs). The split around 500 cm⁻¹ is indicative of crystalline material.

FIG. 4—Time sweeps of GNP-free inorganic control gels composed of sHA-NPs (denoted sHA-NPs) and reference gels composed of needle-shaped CaP nanoparticles (denoted nHA-NPs) at different solid contents.

FIG. 5—Storage moduli (G) of GNP-free inorganic control gels before and after destructive shearing (oscillatory strain sweep with increasing strain from 0.1% to 1000% with a fixed angular frequency of 1 rad/s) and recovery (oscillatory time sweep at 0.5% strain and an angular frequency of 1 rad/s for 300 s). Results indicate a lack of realistic self-healing for these gels.

FIG. 6A—Storage moduli (G) of colloidal composite gels composed of sHA-NPs or nHA-NPs mixed with GNPs as a function of solid content from 10 wt % to 20 wt % and HA-NPs/GNPs weight ratio (R value) of 1.

FIG. 6B—As for FIG. 6A, but for HA-NPs/GNPs weight ratio (R value) of 2.

FIG. 7A—Storage moduli (G′) of colloidal composite gels composed of sHA-NPs and GNPs at a solid content of 20 wt % in different liquid media at sHA-NPs/GNP weight ratio (R value) of 1.

FIG. 78—as for FIG. 7A, but at sHA-NPs/GNP weight ratio (R value) of 2.

FIG. 8A—Storage moduli of colloidal composite gels after 5 cycles of destructive shearing (oscillatory strain sweep with increasing strain from 0.1% to 1000% with a fixed angular frequency of 1 rad/s) and recovery (oscillatory time sweep at 0.5% strain and an angular frequency of 1 rad/s for 300 s). Data for gels with R value 1.

FIG. 8B—representations of G′ values of FIG. 8A.

FIG. 8C—as FIG. 8A but for gels with R value 2.

FIG. 8D—representations of G′ values of FIG. 8C.

FIG. 8E—as FIG. 8A but for gels (15 wt.-%) with R values 1 or 2 as indicated, and sHA particles were Zn-loaded.

FIG. 9—Survival of intracellular S. aureus-mCherry 24 h after addition of NPs. All NPs containing antibacterial agents significantly reduce intracellular S. aureus survival in comparison to the ‘no treatment’ control group. Systemically delivered zinc and vancomycin did not cause a significant reduction in survival of intracellular S. aureus, while using the particles of the invention did reduce survival. LIVE/DEAD staining of the human THP-1 macrophages with the phagocytosed S. aureus after contact with antibacterial agent or nanoparticles for 24 h showed that there is no difference in cell number between the various groups, indicating that the reduced survival of S. aureus was not caused by cytotoxicity.

FIG. 10A—Combinations of VGelA NPs and 20 mol % ZnHA to explore combined effects on the reduction of intracellular S. aureus. Statistics are as compared to a ‘no treatment’ control (1000%). LIVE/DEAD staining showed no cytotoxic effect.

FIG. 10B—as for FIG. 10A but with unloaded nanoparticles (no Zn or Vancomycin). There was no significance compared to the ‘no treatment’ control (100%).

FIG. 11A—Combinations of VGelB NPs and 20 mol % ZnHA to explore combined effects on the reduction of intracellular S. aureus. Statistical analysis was performed relative to a ‘no treatment’ control (100%). LIVE/DEAD staining showed no cytotoxic effect. Notably, when comprised in the tested gels, vancomycin-loaded Gelatin B nanoparticles outperform Gelatin A analogues by achieving a better kill of intracellular bacteria.

FIG. 11B—as for FIG. 11A but with unloaded nanoparticles (no Zn or Vancomycin). There was no significance compared to the no treatment control (100%).

FIG. 12A—Combinations of systemic zinc and vancomycin as control group. Systemic zinc and vancomycin do not decrease intracellular survival for more than 50%. An effective combined effect was only observed for zinc:vancomycin of 300:5.5 μM compared to the ‘no treatment’ control (100%).

FIG. 12B—representative LIVE/DEAD staining of the human THP-1 macrophages with phagocytosed S. aureus-mCherry and in contact with antibacterial agents for 24 h as indicated (numbers are μM). Conditions are indicated above the related micrographs. For vancomycin without Zn, only the highest dose is shown. Lower doses were comparable.

### EXAMPLES

### Example 1—Materials and Methods

### 1.1. Provision of Nanoparticles

### 1.1.1 Gelatine Nanoparticle (GNPs) Synthesis

GNPs were prepared by a desolvation process using ethanol. Briefly, 1.25 g gelatine type A (Gel A, from porcine skin, 300 Bloom, isoelectric point (IEP)~9, provided by Rousselot, Batch No.: A1515110F) was dissolved in 25 mL demineralized water under constant stirring (500 rpm) at 40° C. The pH was lowered to 3.0 using 1 M HCl (37% fuming, Merck) whereafter 100 mL ethanol (Boom) was added dropwise (5 mL/min) under constant stirring (1000 rpm) to induce gelatine desolvation and aggregation into spherical GNPs. The GNPs were crosslinked by addition of 5 mL of 5 mM 1-ethyl-3-(3-dimethyl-aminopropyl)carbodiimide (EDC, Sigma) and 1 mM N-hydroxysuccinimide (NHS, Sigma) dissolved in deionized water under constant stirring (500 rpm) at room temperature. After 16 h of crosslinking, a crossflow setup based on a Sartorius Stedim Sartocon Slice filter holder equipped with a 300 kDa cutoff membrane was used to remove ethanol and wash GNPs. The GNPs were washed with 3×1 L demineralized water and stored as dispersion (30 mg mL⁻¹) in demineralized water at 4° C. A Malvern Zetasizer Lab dynamic light scattering device was used to determine i) the hydrodynamic diameter of GNPs dispersed in demineralized water and ii) the zeta-potential of GNPs dispersed in 5 mM HEPES buffer (Sigma) at pH 7.4. The obtained GNPs had a hydrodynamic diameter of 237.1±2.3 nm with a polydispersity index 0.04±0.02 and a zeta-potential of +18.8±0.6 mV.

### 1.1.1.1 Vancomycin Loading of GNPs

GNPs were prepared by a desolvation process using ethanol. Briefly, 1.25 g gelatin type A or type B (Gel A or Gel B, from porcine skin, 300 Bloom (type A) and 247 Bloom (type B), isoelectric point (IEP)~9 and ~5 respectively, Rousselot, Gent, Belgium) were dissolved in 25 mL demineralized water under constant stirring (500 rpm) at 40° C. The pH was lowered to 3.0 using 1 M HCl (37% fuming, Merck) whereafter 100 mL ethanol was added dropwise (5 mL/min) under constant stirring (1000 rpm) to induce gelatin desolvation and aggregation into spherical Gel NPs. The Gel NPs were crosslinked by the addition of 5 mL of 5 mM, 1-ethyl-3-(3-dimethyl-aminopropyl)carbodiimide (EDC, Sigma-Aldrich) and 1 mM N-hydroxysuccinimide (NHS, Sigma-Aldrich) dissolved in deionized water under constant stirring (500 rpm) at room temperature. After 16 h of crosslinking, a crossflow setup based on a Sartorius Stedim Sartocon Slice filter holder equipped with a 300 kDa cutoff membrane was used to remove ethanol and wash Gel NPs. The Gel NPs were washed with 3×1 L demineralized water and stored as dispersion (30 mg mL⁻¹) in demineralized water at 4° C.

To fabricate vancomycin-loaded GNPs (VGNPs), 100 μg vancomycin (Sigma-Aldrich) was dissolved in 1 mL of a freshly prepared suspension of GNPs in demineralized water (8 mg/mL) and equilibrated at 4° C. for at least 12 h to allow absorption and diffusion of vancomycin to the Gel NPs. Afterwards, the VGNPs were centrifuged at 10.000×g for 10 min to remove unbound vancomycin, after which particles were resuspended in phosphate buffered saline (PBS).

1.1.2 Synthesis of Spherical-Shaped Hydroxyapatite Nanoparticles (sHA-NPs)

sHA-NPs were synthesized by a wet-chemical precipitation method according to the following reaction scheme:

10Ca(CH₃CO₂)₂+6Na₃PO₄+H₂O+4O₂═Ca₁₀(PO₄)₆(OH)₂+18NaCH₃CO₂+3H₂O+4CO₂

Briefly, 100 mL calcium acetate solution (83.5 mM, Sigma) was poured into an aqueous solution of 100 mL trisodium phosphate (50 mM, pH~12.8, Merck) under vigorous stirring for 2 h at 40° C., followed by addition of sodium citrate (940 mg, 18.2 mM) to render the synthesized nanoparticles negatively charged. The reaction was continued for another 18 h to produce crystalline sHA-NPs. sHA-NPs were centrifuged (10000 rpm, Heraeus Multifuge X1R, Thermo Fisher) for 10 min and resuspended in deionized water three times and finally stored as an aqueous suspension (30 mg mL⁻). The resulting sHA-NPs comprise calcium and phosphate and thus are CaP nanoparticles.

### 1.1.2.1 Synthesis of Zinc-Doped Hydroxyapatite Nanoparticles (ZnHA-NPs)

ZnHA NPs with molar ratio [Ca+Zn]/[P]=1.67 were synthesized as follows: a 50 mM aqueous trisodium phosphate solution (Na₃PO₄·12 H₂O; Sigma-Aldrich, Saint Louis, MI, USA) was heated to 60° C. Appropriate amounts of 100 mM zinc nitrate (Sigma-Aldrich) and 83.5 mM calcium acetate monohydrate (Ca(C₂H₃O₂)₂ H₂O; Sigma-Aldrich) solutions were added to reach zinc molar percentages of 0, 10, 15 and 20 mol %. Afterwards, tribasic sodium citrate (Na₃C₆H₅O₇; Merck, Darmstadt, Germany) in a concentration of 9.4 mg/mL was added to stabilize the synthesized NPs. After continuous stirring for 24 h at 1000 rpm at 60° C., the resulting precipitate was centrifuged and washed three times with demineralized water. Finally, the NPs were either stored in suspension at 4° C., or lyophilized for 48 h and stored at room temperature.

1.1.3 Synthesis of Needle-Shaped CaP Nanoparticles (Referred to as nHA-NPs)

For reference experiments, needle-shaped CaP nanoparticles were also prepared. The nHA-NPs were prepared using an established wet-chemical precipitation method based on the reaction between calcium hydroxide and ortho-phosphoric acid.

Briefly, 100 mL H₃PO₄ solution (50 mM) was added dropwise into an aqueous suspension of 100 mL Ca(OH)₂ (83.5 mM) under constant stirring, followed by adjustment of the pH to 9.0 using sodium hydroxide (1 M). After reaction for 2 h at 40° C., sodium citrate (940 mg, 18.2 mM) was added to render the synthesized nanoparticles negatively charged, where after the reaction was continued for 18 h. nHA-NPs were centrifuged (10000 rpm, 5 min) and resuspended in deionized water three times, and finally stored as an aqueous suspension (30 mg mL⁻¹).

### 1.1.4 Fluorescent Labelling of Synthesized Nanoparticles

To allow for the visualization of cellular HA NPs and ZnHA NP uptake, HA NPs and ZnHA NPs were fluorescently labelled with 5-FAM-ZOL dye (a zoledronate-conjugated fluorescein-type dye, Biovinc, Pasadena, CA, USA), 100 μL 0.01 nM FAM-ZOL dye was added to the HA NPs and ZnHA NP (1 mg/ml) suspended in PBS at pH 7.4.

To fluorescently label GelA and GelB NPs, 50 mL of a 0.1 mg/mL Fluorescein 5(6)-isothiocyanate (FITC; Sigma-Aldrich) dissolved in demineralized water was added to the GelA or GelB NPs suspension (10 mg/mL, 200 mL). After an incubation time of 6 h at room temperature, the remaining free FITC was removed via crossflow filtration. For the visualization of internalization of VGelA NPs or VGelB NPs, 100 μg FITC-vancomycin (Sigma-Aldrich) was added in 1 mL suspension of VGelA NPs or VGelB NPs (8 mg/ml) in demineralized water to obtain FITC-VGelA NPs and FITC-VGelB NPs. All labeled NPs were stored in suspension at 4° C. and used without further modification.

### 1.2 Physicochemical Characterization of Nanoparticles

The hydrodynamic diameter of nanoparticles dispersed in deionized water and the zeta-potential of nanoparticles dispersed in 5 mM HEPES buffer at pH 7.4 were characterized using dynamic light scattering using a Zetasizer Lab apparatus (Malvern Instruments Ltd.). The molecular and crystal structure of lyophilized sHA-NPs and nHA-NPs were analysed using attenuated total reflectance Fourier transform infrared spectroscopy (ATR-FTIR, Perkin Elmer) and X-ray diffraction (XRD, Phillips X'Pert, PW3710), respectively. The morphology of the particles was visualized by scanning electron microscopy (SEM, JEOL 6301). The average size of dried particles was determined by averaging the length and width of at least 200 particles using SEM images by digital image analysis software (image J, NIH).

1.3 Self-Assembly of GNPs and sHA-NPs or nHA-NPs (HA-NPs)

The particle size of mixed dispersions of GNPs and HA-NPs in deionized water was monitored by DLS as a function of time. The effect of HA-NPs/GNPs ratio on the self-assembly of oppositely charged GNPs and HA-NPs was investigated at HA-NPs/GNPs ratios of 1 and 2, respectively, followed by monitoring the change of particle size in time using DLS.

### 1.4. Colloidal Composite Gel Preparation and Characterization

### 1.4.1 Colloidal Composite Gel Preparation

Colloidal composite gels were fabricated by thoroughly mixing GNPs (5 mL or 10 mL, 30 mg mL⁻¹) with HA-NPs (10 mL, 30 mg mL⁻¹) colloidal suspensions. The GNPs and HA-NPs coagulated immediately after mixing, whereafter the dispersions were equilibrated for 1 h before being freeze-dried for 48 h. Colloidal gels were prepared at different total solid content (10 wt %, 15 wt % and 20 wt % at HA-NPs/GNPs weight ratios of 1 and 2, respectively, in deionized water to compare their rheological performance. The 20 w/w % colloidal gels were further prepared in 1×PBS (Gibco™, Thermo Fisher) and cell culture medium (alpha-MEM, Gibco™, Thermo Fisher) to compare the influence of the ionic strength of the liquid phase on rheological properties.

### 1.4.2 Physicochemical Characterization of Colloidal Composite Gels

The surface morphology and inner microarchitecture of the colloidal gels were assessed by scanning electron microscopy. Freshly prepared colloidal gels were flash frozen in liquid nitrogen and freeze-dried. Afterward, samples were sputter-coated with Au/Pd and imaged in an analytical scanning electron microscope (SEM, JEOL 6301). The assembled colloidal gel network morphological features and particle distribution were further evaluated through confocal laser scanning microscopy. Fluorescently labeled nanoparticle building blocks were produced by loading FITC (ThermoFisher Scientific) and BoneTag™ (IRDye® 680RD, LI-COR® Biosciences) fluorescent probes in GNPs and HA-NPs, respectively. Colloidal gels were imaged in a high-resolution LSM900 confocal laser scanning microscope (Zeiss) equipped with a 20×/NA 0.8 objective, GaAsP/PMT detectors. All data were postprocessed in Imaris software (v9) and Zeiss ZEN v2.3 blue edition software (Carl Zeiss Microscopy GmbH).

### 1.4.3 Rheological Characterizations

The viscoelastic properties of the colloidal gels were characterized at different solid content and in different liquid media using a rheometer (AR2000ex, TA Instruments). Oscillatory time, frequency and strain sweeps confirmed that all rheological characterizations were performed within the linear viscoelastic region. All measurements were performed using a flat steel plate geometry (8 mm diameter) and 0.5 mm gap size at 25° C. The storage modulus G′, loss modulus G″ and tan(delta) were determined using an oscillatory time sweep test for 5 min at a constant strain of 0.5% and angular frequency of 1 rad/s.

### 1.4.4 Characterization of Self-Healing Capacity

The self-healing properties of colloidal gels were studied in a strain range from 0.1-1000% at constant frequency of 1 rad/s at 25° C., to mimic the high shear forces upon extrusion through narrow needles. The viscoelastic parameters (G′ and G″) of GNPs/HA-NPs colloidal composite gels were measured at different solid content and in different liquid media as a function of time by means of oscillatory time sweeps (5 min, 1% strain, 1 rad/s angular frequency) before and after severe destruction of the gel network (1000% strain, 1 min, 1 rad/s angular frequency).

### 1.4.5 Stress Relaxation

The stress relaxation of hydrogels was determined in step-strain experiments using a previously established protocol that allows stress relaxation measurements at stepwise increasing strain on the same hydrogel sample. In brief, a step-strain was performed within a strain-raise time of 0.2 s, whereafter the strain was kept constant for 10 min to measure relaxation. After the relaxation phase, the geometry was slowly turned back (negative strain) until stress was 0. After a 10 min equilibration phase, the next step-strain at higher strain was performed. This protocol allowed measurement of stress relaxation at stepwise increasing strain on the same sample without cumulative stress build-up, given that the hydrogels are self-healing at the tested strains.

### 1.4.6 Colloidal Gel Swelling Analysis

The swelling of colloidal gels was quantified by gelling 100 μL, GNPs/HA-NPs at different solid content in PBS (n=3) and covering them with 1 mL, 1×PBS and gravimetric measurement of swelling after storage at different time points (e.g. 1, 3, 5 and 7 days) at 37° C.

### 1.5. Internalization of Nanoparticles in Human THP-1 Macrophages

### 1.5.1 Cell Culture

For cell culture experiments, human THP-1 macrophages (American Type Culture Collection, Manassas, USA) were maintained in Roswell Park Memorial Institute (RPMI) 1640 media supplemented with 2 mM Glutamine (GIBCO, New York, NY, USA) and 100/fetal bovine serum (FBS, Sigma-Aldrich). The human THP-1 macrophages were sub-cultured once a week and incubated at 37° C. in a humidified atmosphere containing 5% CO₂. The human THP-1 macrophages were passaged by centrifugation when reaching a confluency of 10⁶ cells/mL. In the following experiments, cell seeding is performed with media enriched with 50 ng/ml phorbol 12-myristate 13-acetate (PMA; Sigma-Aldrich) to allow for adherence of cells over 48 h.

### 1.5.2 Internalization of Nanoparticles

Human THP-1 macrophages were cultured as described above and seeded with a density of 200.000 cells/cm² in an 8-well μ-slide (ibidi, Gräfelfing, Germany) for 48 h. Labeled NPs were diluted in RPMI cell culture medium to reach a final concentration of 50 μg/mL. Afterwards 200 μL particle suspension was added to the human THP-1 macrophages. As a control group, 5 μL of dissolved FITC-vancomycin (100 μg/ml) was added, while, human THP-1 macrophages were cultured with RPMI media without NPs serving as a negative control. The next day, cells were washed twice with PBS and stained with 1 μM CellTrace yellow (Invitrogen, Waltham, MA, USA) in PBS according to the manufacturer's instructions. Additionally, lysosomal compartments were stained with 50 nM LysoTracker deep red (Thermo Fisher, Waltham, MA, USA) in phenol-red-free RPMI medium supplemented with 10% FBS and 20 mM HEPES 30 min prior to imaging. The internalization of the labelled NPs and FITC-vancomycin was visualized using a Leica TCS SP8 SMD confocal microscope (Leica Microsystems, Wetzlar, Germany), equipped with an HCX PL APO 63×/0.40 water immersion objective and a temperature-controlled stage at 36.5° C. The image analysis software Fiji was used for the reconstruction of images, and the plugin “co-localization finder” was used to visualize the overlaying signal in white and quantify the fluorescence overlay of different channels.

1.6. Co-Culture Model of Phagocytosed S. aureus
1.6.1 In Vitro Phagocytosis of S. aureus-mCherry

S. aureus strain RN4220 expressing mCherry fluorescent protein (S. aureus-mCherry) was used for in vitro studies. Bacterial inoculums were prepared by culturing S. aureus-mCherry in tryptic soy broth (TSB; BD Difco, Leeuwarden, the Netherlands) supplemented with 10 μg/mL chloramphenicol (Merck) shaking at 120 rpm and 37° C. to the mid logarithmic growth phase. S. aureus-mCherry bacteria were pelleted by centrifugation (3000 g, 10 min), re-suspended in 1 mL of NaCl and 0.25 mL human serum (H1 serum, Bio Whittaker, MD, USA) and incubated for 20 min. The inoculum was adjusted to 10⁸ CFU/mL with RPMI based on the optical density value at wavelength 620 (OD₆₂₀). The human THP-1 macrophages were seeded as described above in 96-well plates at a concentration of 100.000 cells/cm². After 48 h, the media was replaced by 1 μL of the bacterial inoculum (bacteria to cell ratio of 1:1) and incubated for 45 min at 37° C. and 5% CO₂. Afterwards, the human THP-1 macrophages were washed three times with 50 μL PBS and one time with 200 μL PBS to prevent carry-over of planktonic S. aureus-mCherry. RPMI media supplemented with 5 μg/mL gentamycin (Sigma-Aldrich) was added to control the extracellular growth of S. aureus-mCherry.

### 1.6.2 Validation of the Co-Culture Model

Human THP-1 macrophages were seeded in a concentration of 100.000 cells/cm² in 8-well μ-slide (ibidi, Gräfelfing, Germany) and were allowed to phagocytose S. aureus-mCherry as described above. After 24 h, the human THP-1 macrophages were washed twice with PBS and lysosomal compartments were stained with 50 nM LysoTracker deep red as described above. The internalization of the S. aureus-mCherry was visualized using a Leica SP8-X DLS Lightsheet microscope (Leica Microsystems, Wetzlar, Germany). The image analysis software Fiji was used for the reconstruction of images, and the plugin “co-localization finder” was used to quantify the fluorescence overlay of different channels. The overlaying signal is also visualized in white.

Additionally, a LIVE/DEAD cell viability assay (Invitrogen, Waltham, MA, USA) was used according to the manufacturer's instructions to visualize the cells after 24 h of phagocytosis. In brief, 100 μL of 4 μM Ethidium homodimer 1 and 2 μM calcein AM solution in PBS was added to the human THP-1 macrophages and incubated for 30 min before visualization using a Leica SP8-X DLS Lightsheet microscope (Leica Microsystems, Wetzlar, Germany). Human THP-1 macrophages without phagocytosed S. aureus-mCherry served as negative control. The amount of living cells were quantified by counting using three LIVE/DEAD images and compared between macrophages with and without phagocytosed S. aureus.

1.6.3 Visualization of Intracellular S. aureus-mCherry and Nanoparticles

Human THP-1 macrophages were cultured at a concentration of 100.000 cells/cm² in 8-well μ-slide (ibidi, Gräfelfing, Germany) and were allowed to phagocytose S. aureus-mCherry as described above. After phagocytosis, RPMI supplemented with 5 μg/ml gentamycin was enriched with 50 μg/ml fluorescently labelled NPs. The cells were incubated for 24 h whereafter lysosomal compartments were stained with 50 nM LysoTracker deep red as described above. The S. aureus-mCherry, lysosomes and labelled NPs were visualized by confocal laser scanning microscopy. The image analysis software Fiji was used for the reconstruction of images, and the plugin “co-localization finder” was used to visualize the overlaying signal in white and quantify the fluorescence overlay of different channels.

### 1.6.4 Intracellular Antibacterial Activity of Nanoparticles

The human THP-1 macrophages were allowed to phagocytose S. aureus-mCherry as described above. To investigate the intracellular killing efficiency of the synthesized NPs, RPMI media with 5 μg/ml gentamycin was enriched with 50 μg/ml of synthesized NPs for 24 h. Moreover, to assess the synergistic effect of zinc and vancomycin, a combination of 20 mol % ZnHA and VGelA NPs was added to the co-culture in different concentrations. After 24 h, the human THP-1 macrophages were lysed with 100 μl of 0.025% TritonX-100 to determine the amount of intracellular S. aureus-mCherry number. After lysis, the solution was serial diluted in PBS and spot-plated on agar plates. As control groups, dissolved zinc (300 μM) and dissolved vancomycin (5.5 μM) were used. Additionally, the viability of the THP-1 macrophages is assessed using a LIVE/DEAD cell viability assay (Invitrogen, Waltham, MA, USA) as mentioned above. Images were obtained with a Leica SP8-X DLS Lightsheet microscope (Leica Microsystems, Wetzlar, Germany). Human THP-1 macrophages without phagocytosed S. aureus-mCherry served as negative control.

### 1.6.5 Statistical Analysis

All data were presented as mean±standard deviation (SD) of samples in triplicate (n=3), unless mentioned otherwise. Statistical differences were evaluated using Prism (GraphPad software, San Diego, CA). Statistical comparison of data from different compositions was carried out using one-way analysis of variance (ANOVA) with additional multiple comparison test (Tukey) for differences between groups.

### Example 2—Characterisation of Nanoparticles

2.1 Characterization of sHA/nHA-NPs

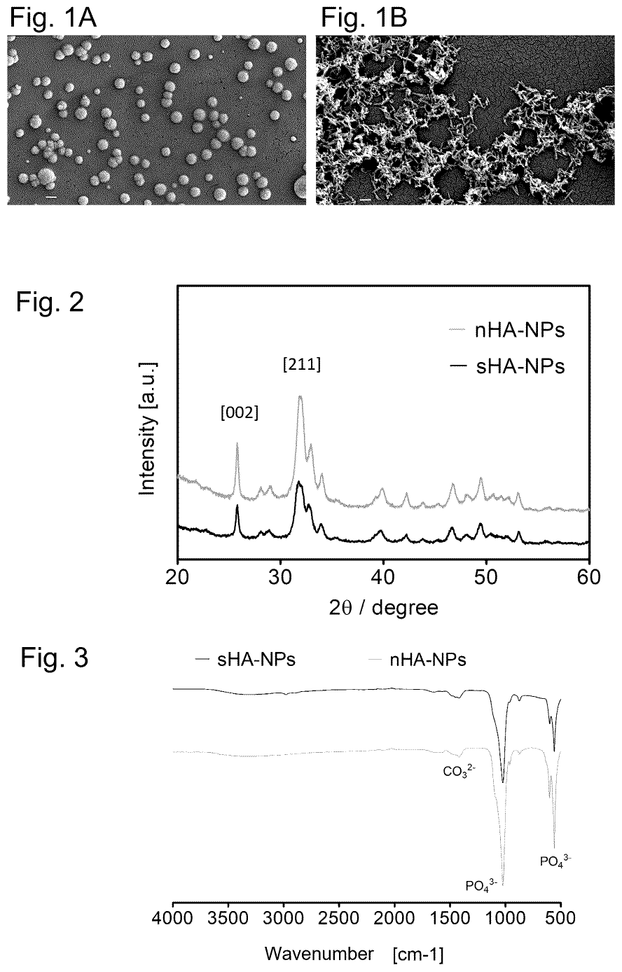

The one-pot wet-chemical precipitation method resulted in the formation of spherical-shaped HA nanoparticles with an average size of 219.3±2.7 nm. The zeta-potential values of sHA-NPs dispersed in pure water and 5 mM HEPES buffer were −24.9±0.2 and −19.5±0.4 mV (Table S2.1), respectively. The effective adsorption of citrate anions onto HA nanoparticles rendered particles negatively charged. The particle size was constant in time, as confirmed by overnight monitoring of particle size within the suspensions using DLS, indicating that the repulsive electrostatic forces between negatively charged sHA-NPs contributed to preventing agglomeration and sedimentation. This was further confirmed by the homogeneous size distribution shown by SEM imaging (FIG. 1A). The needle-shaped CaP particles (nHA-NPs) aged for 20 h resulted in an average length of 166.8±29.7 nm and a width of 27.6±5.8 nm (Table S2.1). In contrast, the addition of sodium citrate to the suspension containing nHA-NPs did not substantially enhance the negative charge of the particles (−11.9±1.3 mV in pure water and −9.2±1.2 mV in 5 mM HEPES buffer). Due to this relatively low particle charge of needle-shaped CaP particles, these particles agglomerated considerably, as evidenced by their size of about 4.72±0.89 μm) as measured using DLS, which resulted in sedimentation of nHA-NPs after 120 s of equilibration during the zeta-potential measurement. The SEM images of nHA-NPs in FIG. 1C and FIG. 1D also confirmed this aggregation of needle-shaped nanoparticles, which was also caused by the relatively low particle charge of the CaP needles in suspension. In similar experiments, spherical particles without citrate were also found to somewhat cluster.

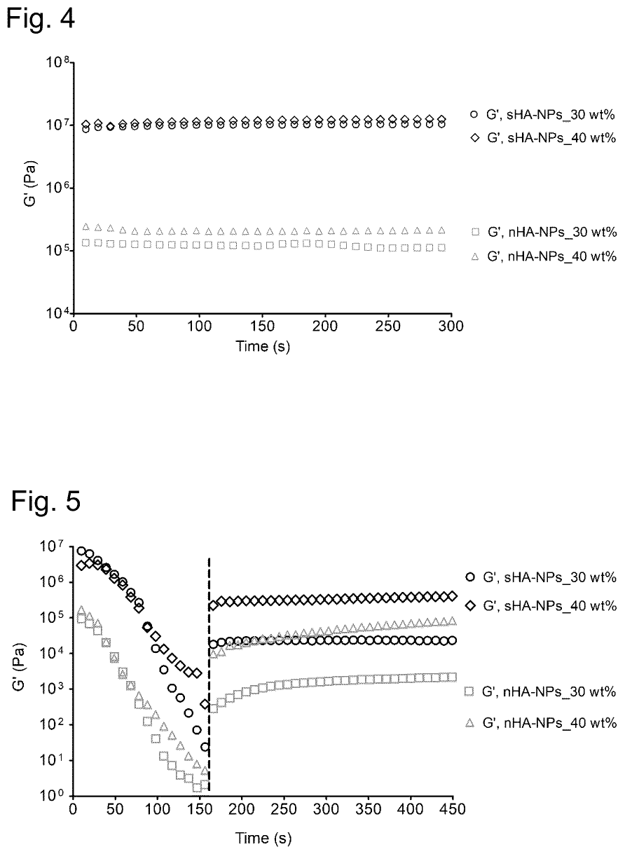

The XRD patterns of the HA-NPs is shown in FIG. 2. The diffraction peaks at 2θ=25.9°, 31.9°, 32.9°, 34.1°, 39.9°, 46.6°, and 49.5° corresponded to the (002), (211), (300), (202), (310), (222), and (213) planes of HA, respectively. The broad reflection peak as observed in the XRD patterns confirmed the poorly crystalline apatitic crystal structure of the precipitated HA-NPs, while the adsorption of citrate anions did not affect the XRD pattern of the HA-NPs.

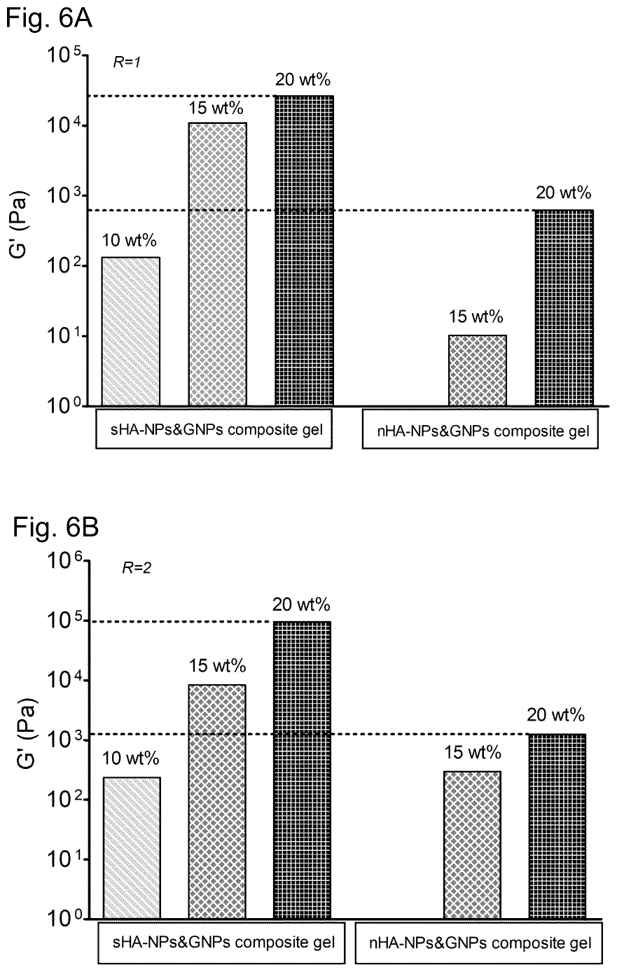

Molecular groups corresponding to hydroxyapatite were identified by FTIR spectroscopy. FIG. 3 illustrates the FT-IR spectrum of HA-NPs crystals with two different shapes. A broad absorption peak near 3315 cm⁻¹ corresponded to water adsorbed on the surface of the HA nanoparticles, while the vibration peaks at 566, 605, 963, and 1029 cm⁻¹ were attributed to the asymmetric stretching vibration peaks of P—O in the PO₄³⁻ groups. Absorptions corresponding to B-type carbonate (CO₃²⁻ bending vibrations) were observed at 876 cm-1 in combination with and CO₃²⁻ stretching vibrations at 1420 and 1456 cm⁻¹. XRD and FT-IR spectroscopy results showed that both types of HA-NPs were phase-pure.

In table S2.1 below, data are shown for the spherical-shaped HA nanoparticles and needle-shaped CaP nanoparticles, showing their hydrodynamic size as measured using DLS, dry particle size obtained using SEM, particle polydispersity (PI), and zeta-potential in deionized water or 5 mM HEPES buffer at pH 7.4.

TABLE S2.1
Overview of nanoparticles.

| | Size | | Size | Zeta | Zeta |
|---|---|---|---|---|---|
| | (DLS, nm) | PDI | (SEM, nm) | (dH₂O mV) | (HEPES, mV) |
| sHA-NPs | 219.3 ± 2.7 | 0.19 ± 0.01 | 173.0 ± 28.8 | −24.9 ± 0.2 | −19.5 ± 0.4 |
| (citrate) | | | | | |
| sHA-NPs | | 0.16 ± 0.10 | | 0.63 ± 0.27 | |
| (no citrate) | | | | | |
| nHA-NPs | 4711.3 ± 892.2 | 1 | 166.8 ± 29.7 (L) | −11.9 ± 1.3 | −9.2 ± 1.2 |
| (citrate) | | | 27.6 ± 5.8 (W) | | |

### 2.2 Spherical HA Nanoparticles Yield Improved Colloidal Composite Gels

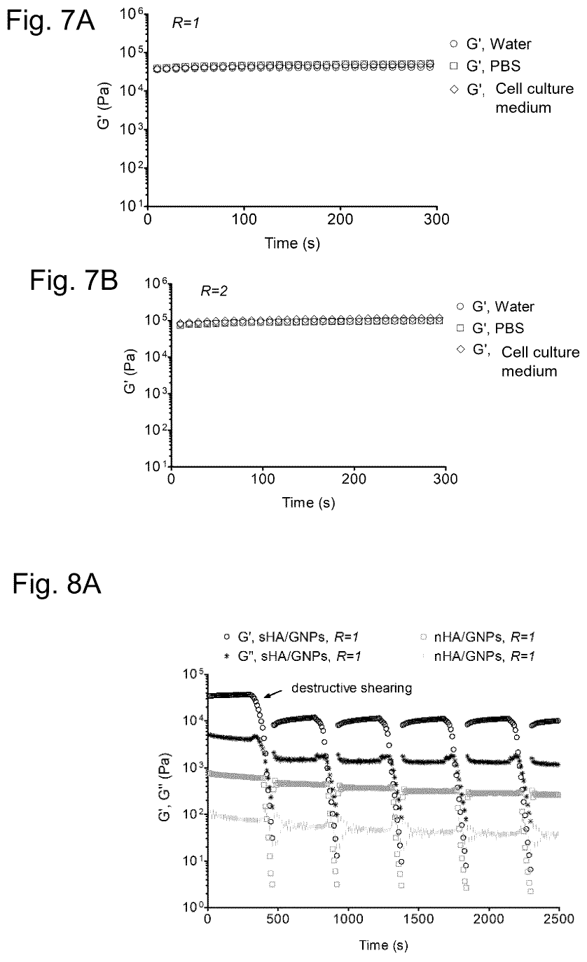

GNP-free inorganic control gels made of sHA-NPs or nHA-NPs were elastic characterized by high G′ values at solid contents of 30 wt % and 40 wt %, respectively (FIG. 4). At similar solid content, spherical-shaped HA-NPs formed colloidal gels which revealed considerably higher storage moduli (~10.34 MPa & ~12.37 MPa) than colloidal gels composed of needle-shaped CaP nanoparticles (~0.19 MPa & ~0.21 MPa).

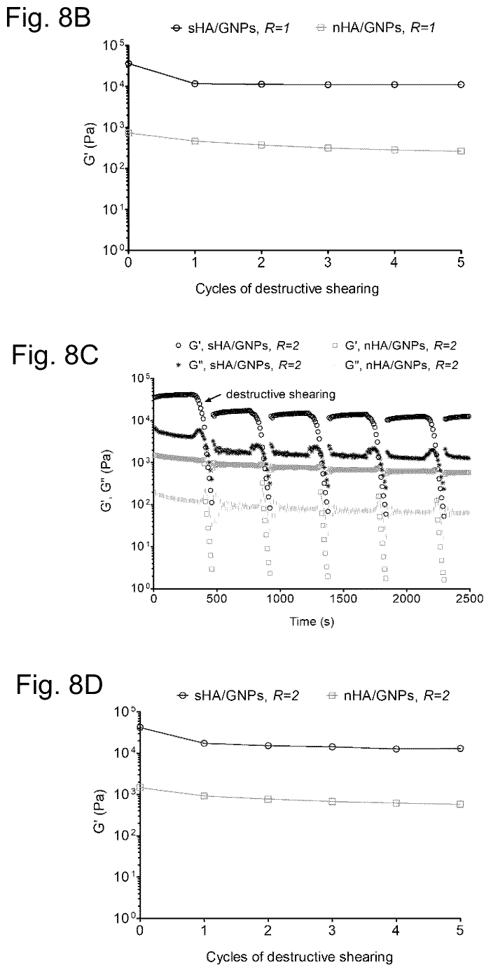

Generally, the reversibility of the noncovalent interparticle bonds often endows colloidal gels assembled from nanoparticles with self-healing properties. The recovery of the storage modulus after shear-induced gel network destruction (0.1-1000% strain), did not reveal high substantial self-healing capacity for purely inorganic HA-NPs gels (<50% self-healing), irrespective of their particle shape (FIG. 5).

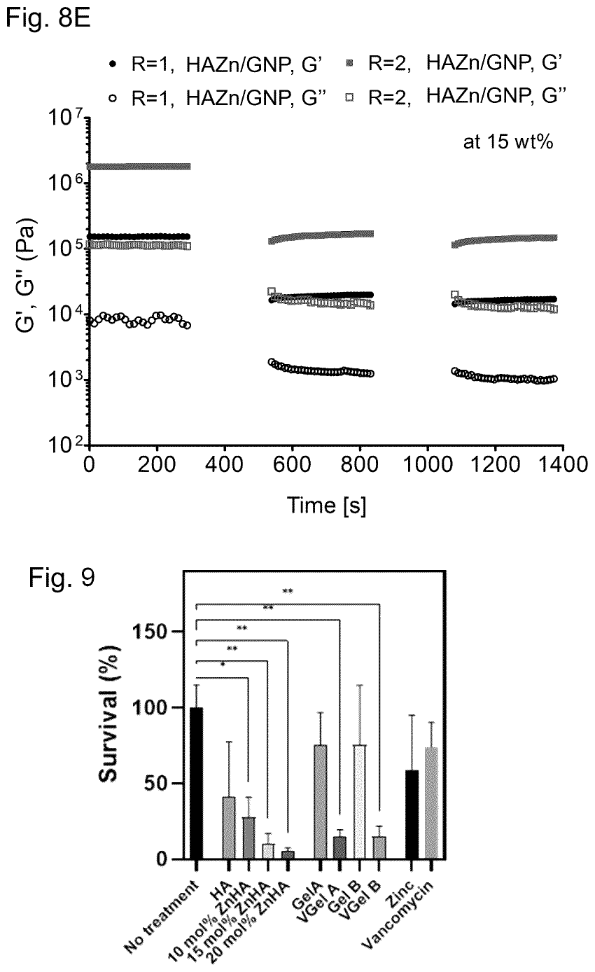

Colloidal composite gels composed of GNPs and either spherical-shaped HA-NPs or needle-shaped CaP nanoparticles were subjected to extensive rheological characterizations. These colloidal composite gels were rather elastic and self-healing colloidal composite gels, which may benefit their injectability/printability/moldability for biomedical applications. These rheological tests were performed at HA-NPs/GNPs ratios (R) of 1 and 2. From FIG. 6A and FIG. 6B it can be concluded that stronger networks were formed with increasing R upon addition of stiff HA nanoparticles, irrespective of the shapes of the HA nanoparticles. However, storage moduli (G′) were considerably lower (between 28-1000 folds at different solid contents and R values, see FIG. 6A and FIG. 6B) for composite gels composed of needle-shaped CaP nanoparticles at both R values, confirming that at similar solid content colloidal composite gels comprising needle-like nanoparticles were much less cohesive and elastic than composite gels comprising spherical nanoparticles. The 10 wt % composite gels comprising needle-shaped nanoparticles cannot be formed at both R values (G′<G″) These striking differences suggest that nanoparticle shape and dispersity strongly affect the viscoelastic behaviour of colloidal composite gels comprising these nanoparticles, where spherical nanoparticles of the invention produce gels with strongly improved properties.

### 2.3 Improved Gels are Robust to Ionic Strength of the Liquid Media

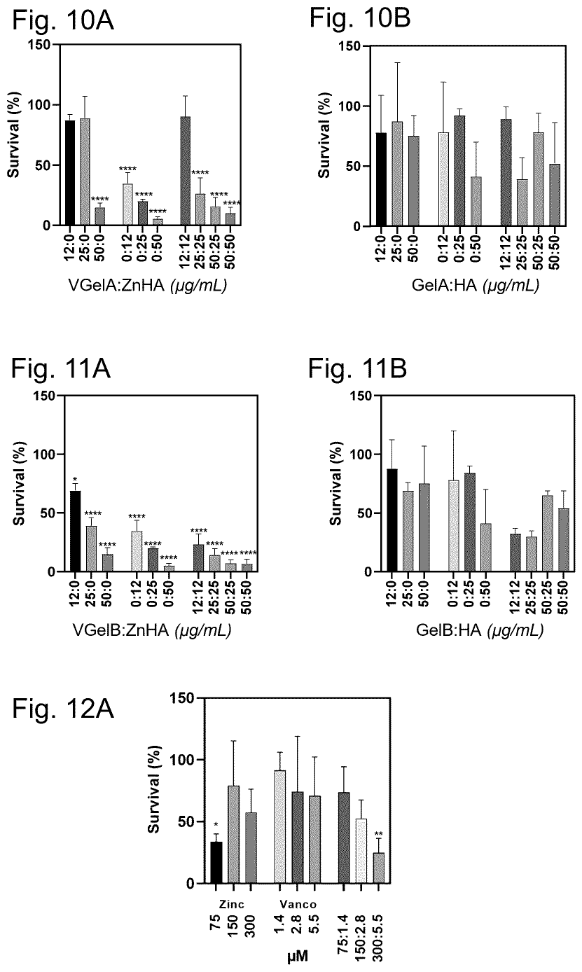

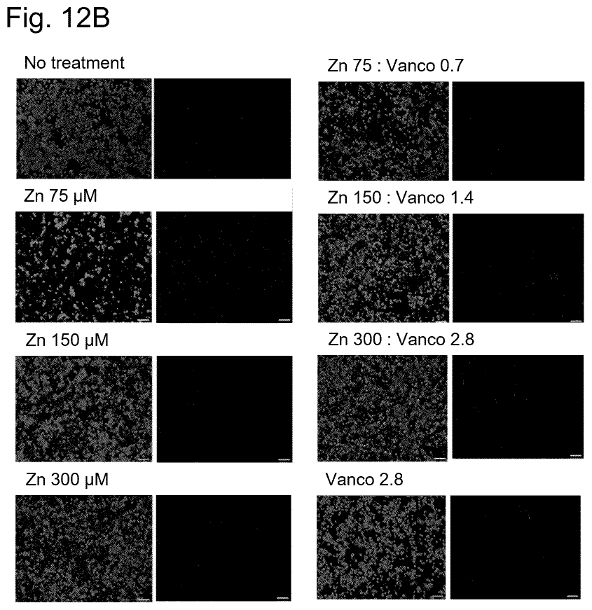

Since electrostatic interactions between charged nanoparticles strongly depend on the ionic strength of liquid media, the inventors investigated the influence of several liquid media on the viscoelastic properties of colloidal composite gels, i.e., deionized water (G′, 42.1 KPa at R=1 & 96.9 KPa at R=2), 1×PBS (G′, 50.9 KPa at R=1 & 98.1 KPa at R=2) and α-MEM (G′, 49.1 KPa at R=1 & 116.0 KPa at R=2). Generally, the ionic strength did not affect the viscoelastic parameters of colloidal composite gels composed of sHA-NPs and GNPs at a solid content of 20 wt % at R values of 1 and 2 (FIG. 7A and FIG. 7B).

### 2.4 Spherical HA Nanoparticles Yield Improved Colloidal Composite Gels

The robustness to repeated shearings of colloidal composite gels comprising spherical-shaped vs. needle-shaped CaP nanoparticles was investigated by severely destructing (0.1-1000% strain) the gels for multiple cycles and monitoring the recovery of gel strength per consecutive destruction cycle (G′). FIG. 8A through FIG. 8D display a strong capacity of sHA-NPs/GNPs composite gel at solid content of 20 wt % for self-healing characterized by 96%-100% recovery after the first destruction cycle, irrespective of the R value. In contrast, the nHA-NPs/GNPs gels showed a gradual decay in G′ as function of destruction cycles, corresponding to liquid-like behaviour. Relevant parameters are shown in Table S2.4, where the storage modulus (G′) after each cycle of destructive shearing between sHA-NPs/GNPs & nHA-NPs/GNPs composite gels at R values of 1 and 2 is shown. The solid content of both composite gels is 20 wt %.

TABLE S2.4
Overview of the storage modulus (G′) after destructive shearing

| Entry | | 0 | 1 | 2 | 3 | 4 | 5 |
|---|---|---|---|---|---|---|---|
| R = 1, G′, | sHA-NPs/GNPs | 36.65 | 11.77 | 11.34 | 11.11 | 11.18 | 11.16 |
| KPa | nHA-NPs/GNPs | 0.78 | 0.52 | 0.42 | 0.34 | 0.28 | 0.26 |
| R = 2, G′, | sHA-NPs/GNPs | 42.02 | 17.27 | 15.20 | 14.21 | 12.61 | 12.98 |
| KPa | nHA-NPs/GNPs | 1.48 | 0.92 | 0.77 | 0.67 | 0.62 | 0.58 |

Loaded gels also display these good properties. FIG. 8E shows storage moduli of Zn-loaded sHA particles in colloidal composite gels with GelA nanoparticles after cycles of destructive shearing, similar to what is shown in FIG. 8A-D for unloaded gels. The presence of the active agent was found to not negatively impact the good rheological properties.

When a gel is injected, such as injection into a subject, the passage through a syringe can be seen as an extrusion. Thus in practice the relevant parameter is the capacity to maintain a constant level after a first shearing. These results show that the morphology of the HA nanoparticles improves the behaviour of gels in response to multiple shearings, rendering the gels robust to mechanical attack and destructive shear.

### 2.5 Discussion

It can be concluded that colloidal composite gels comprising spherical-shaped HA nanoparticles exhibit favorable viscoelastic behavior in terms of enhanced elasticity, cohesion, and self-healing capacity that manifests as robustness to mechanical attack and repeated destructive shear. The beneficial properties are compared to conventional composite gels that comprise needle-like CaP nanoparticles. The formation of spherical shaped hydroxyapatite nanoparticles with controllable size, aspect ratio, and crystallinity was achieved using an attractive and convenient one-pot aqueous precipitation method. The calcium salts such as acetate and phosphate salts such as trisodium phosphate at different precursor addition speed, pH, and temperature at a fixed Ca/P molar ratio of 1.67 were studied. Spherical hydroxyapatite nanoparticles could be reliably provided and their surprising properties qualify them as an attractive material.

### Example 3—Biological Application of Nanoparticles

### 3.1 Internalization of Nanoparticles

The internalization and intracellular localization of the synthesized NPs were evaluated using confocal microscopy in which the cell cytoplasm, lysosomes and NPs were represented in yellow, red, and green, respectively. The following overlay percentages were found:

TABLE S3.1
colocalization of nanoparticles

| | NPs: | | | | | | | | |
|---|---|---|---|---|---|---|---|---|---|
| | HA NPs (mol % Zn indicated) | | | | Gel NPs (V is FITC-V) | | | | No NPs |
| | Load: | | | | | | | | |
| | 0% | 10% | 15% | 20% | GelA | GelB | VGelA | VGelB | FITC-V |
| Overlay (%) | 74 ± 13 | 85 ± 9 | 89 ± 2 | 89 ± 4 | 93 ± 3 | 93 ± 3 | 89 ± 1 | 93 ± 1 | 0 |

There was a significant difference between HA compared to GelA, GelB and VGelB (p<0.05). Additionally, the “no NPs” highlights the inability of FTIC-vancomycin to be internalized by human THP-1 macrophages. However, when utilizing GelA or GelB NP carriers, vancomycin becomes detectable within human THP-1 macrophages.

3.2 Co-Culture Model of Phagocytosed S. aureus

### 3.2.1 Validation of the Co-Culture Model

A model consisting of human THP-1 macrophages and S. aureus-mCherry was established by facilitating the phagocytosis of S. aureus-mCherry into the human THP-1 macrophages. To investigate the success of this process, confocal microscopy and LIVE/DEAD staining were employed. The cells were visualized using brightfield, while lysosomes and S. aureus-mCherry were highlighted in magenta and red, respectively. The observed degree of overlay between the lysosomes and S. aureus-mCherry bacteria was as high as 95% indicating that the bacteria are internalized within human THP-1 macrophages through an uptake mechanism in lysosomes.

To assess the viability of human THP-1 macrophages after the internalization of S. aureus-mCherry, a LIVE/DEAD staining was conducted. Living cells were marked with green staining, while dead cells were identified with red staining. A comparative analysis of human THP-1 macrophages without phagocytosed bacteria, alongside those containing internalized S. aureus-mCherry, reveals non-significant differences in living cell number (182±8 versus 203±23, respectively). This observation indicates that the human THP-1 macrophages withstand the internalization process of S. aureus-mCherry, thereby affirming the suitability of this model for further investigations.

3.2.2 Visualization of Intracellular S. aureus-mCherry and Nanoparticles

To localize the S. aureus-mCherry and NPs after 24 h of culture, confocal microscopy was employed in which THP-1 macrophages were visualized in brightfield while lysosomes, NPs, and S. aureus-mCherry were respectively indicated by magenta, green, and red. All NPs demonstrated overlay with S. aureus-mCherry indicating co-localization within the human THP-1 macrophages. Overlay was best for gelatin NPs and for ZnHA NPs wherein Zn-content was over 10 mol-%.

TABLE S3.2.2
colocalization of nanoparticles

| | NPs: | | | | | | | |
|---|---|---|---|---|---|---|---|---|
| | HA NPs (mol % Zn indicated) | | | | Gel NPs (V is FITC-V) | | | |
| | Load: | | | | | | | |
| | 0% | 10% | 15% | 20% | GelA | GelB | VGelA | VGelB |
| Overlay (%) | 73 ± 9 | 77 ± 7 | 88 ± 3 | 89 ± 1 | 87 ± 4 | 86 ± 8 | 81 ± 6 | 86 ± 3 |

### 3.3 Intracellular Antibacterial Activity of Nanoparticles

To investigate the intracellular antibacterial efficacy of the synthesized NPs, cellular lysis was performed, and the intracellular S. aureus-mCherry bacteria were subsequently spot plated (FIG. 9). Counting of CFU was conducted to quantify bacterial viability. Notably, spot plating of the culture media surrounding the cells revealed the absence of extracellular bacteria in all groups after 24 h of culture (results not shown). This observation indicates that all the plated bacteria after cell lysis were indeed intracellular and not a result of extracellular bacteria adhering to the external surface of the cells. FIG. 9 shows the impact of the synthesized NPs on the survival of the intracellular S. aureus-mCherry. Comparing the outcomes of HA, 10 mol % ZnHA, 15 mol % ZnHA and 20 mol % ZnHA in relation to the untreated control group, it becomes evident that all zinc doped NPs induce a significant reduction (p<0.01) in the survival of S. aureus-mCherry, with percentages above 10 mol % showing particularly good effects.

Introducing GelA, GelB, VGelA and VGelB NPs into the system yielded no statistical significant outcome between GelA and GelB NPs and the control group, but significant differences were observed between VGelA and VGelB NPs and the control group (p<0.01) compared to the ‘no treatment’ control group, confirming the antibacterial effect of vancomycin. Due to the similar observations of VGelA and VGelB NPs, both types of drug-loaded nanoparticles were used for exploration of a potential synergistic effect of combined zinc and vancomycin delivery. A LIVE/DEAD assay showed the viability of the human THP-1 macrophages after phagocytosis and NP addition after 24 h. Images showed no obvious cell toxicity with the combination of S. aureus-mCherry internalization and NP internalization.

Various concentrations of 20 mol % ZnHA NPs and either VGelA (FIG. 10) or VGelB NPs (FIG. 11) were added to the co-culture of human THP-1 macrophages and intracellular S. aureus-mCherry. LIVE/DEAD images showed no toxicity of the added combinations of NPs or antibacterial agents to the human THP-1 macrophages. It was found that coadministration was viable for each type of gelatin using this gel system. Surprisingly, gelatin B particles were superior, an effect that was markedly pronounced at lower doses.

Systemic zinc and vancomycin were added to the co-culture model (FIG. 12) to observe the relevance of the nanoparticle carriers. It can be observed that all systemically delivered zinc and vancomycin concentrations did not induce a reduction of intracellular S. aureus survival percentages, except for 75 μM zinc and the combination of 300 μM zinc with 5.5 μM vancomycin. Observation of the LIVE/DEAD images indicated that zinc in concentration 75 μM visually decreased the amount of alive cells which might result in the decreased intracellular survival of S. aureus. Thus the gel as a delivery platform could reduce side effects.

1. Method for producing spherical hydroxyapatite nanoparticles having an average size of 60-500 nm, the method comprising the steps of:
i) providing an aqueous calcium salt composition comprising calcium acetate;
ii) providing an aqueous phosphate composition;
iii) combining the aqueous calcium salt composition with the aqueous phosphate composition to obtain a reaction mixture wherein spherical hydroxyapatite (sHA) nanoparticles are formed; and optionally
iv) purifying the sHA nanoparticles.

2. The method according to claim 1, wherein the aqueous calcium salt composition is an aqueous solution or suspension comprising 30 to 150 mM calcium salt.

3. The method according to claim 1, wherein the spherical hydroxyapatite nanoparticles do not comprise organic polymers.

4. The method according to claim 1, wherein the aqueous phosphate composition is an aqueous solution or suspension comprising 20 to 100 mM phosphate, or wherein the phosphate composition comprises about 40-80 mol % phosphate relative to the amount of calcium in the calcium salt composition.

5. The method according to claim 1, wherein the phosphate is phosphoric acid, a dihydrogen phosphate salt, a hydrogen phosphate salt, or a phosphate salt.

6. The method according to claim 1, wherein the reaction mixture is allowed to react for at least 2 hours, and/or wherein the reaction mixture is at a temperature of about 30 to 50° C., and/or wherein sodium citrate is added to the reaction mixture after about 2 hours of reacting.

7. The method according to claim 1, wherein the spherical hydroxyapatite nanoparticles are purified by one or more of centrifugation, filtration, decantation, or resuspension.

8. The method according to claim 1, wherein the spherical hydroxyapatite nanoparticles have an average size of about 100-350 nm, or wherein the zeta potential of the spherical hydroxyapatite nanoparticles is in the range of −30 to −15 mV.

9. The method according to claim 1, wherein the spherical hydroxyapatite nanoparticles are crystalline.

10. Composition comprising spherical hydroxyapatite nanoparticles having an average size of 100-350 nm.

11. Composition according to claim 10, wherein the composition is an aqueous suspension comprising 10-100 mg/mL of the nanoparticles.

12. (canceled)

13. Colloidal composite gel comprising
i) organic nanoparticles; and
ii) spherical hydroxyapatite nanoparticles.

14. Gel according to claim 13, wherein the organic nanoparticles and the spherical hydroxyapatite nanoparticles are present in a weight ratio of 1:10 to 10:1, respectively, or wherein the gel has a total solid content of about 5-50 wt. %.

15. Gel according to claim 13, wherein the storage modulus (G′) of the gel is above 1.

16. (canceled)

17. Gel according to claim 13, comprising
i) gelatin nanoparticles; and
ii) spherical hydroxyapatite (sHA) nanoparticles,
wherein at least one of the gelatin nanoparticles and the sHA nanoparticles comprises an antibiotic agent.

18. The gel according to claim 17, wherein the sHA nanoparticles comprise an antibiotic metal ion.

19. The gel according to claim 17, wherein the gelatin nanoparticles comprise vancomycin and wherein the sHA nanoparticles comprise zinc.

20. The gel according to claim 17, wherein the gelatin nanoparticles comprise gelatin with an isoelectric point of about 4.7-5.5 and comprise vancomycin, and wherein the sHA nanoparticles comprise 12-20 mol % zinc.

21. Composition according to claim 10, wherein the zeta potential of the spherical hydroxyapatite nanoparticles is in the range of −30 to −15 mV.

22. Gel according to claim 15, wherein the storage modulus of the gel is above 2 kPa after 1, 2, 3, 4, or 5 cycles of destructive shearing.
